(19) United States
(12) Patent Application Publication
(10) Pub. No.: US 2017/0362588 A1
(43) Pub. Date: Dec. 21, 2017

(54) SYSTEMS, DEVICES, KITS AND METHODS FOR INDIRECT TRANSFECTION OF MULTIPLE SETS OF NUCLEIC-ACIDS AND TRANSFER OF MOLECULES

(71) Applicant: NOVELLUSDX LTD., Jerusalem (IL)

(72) Inventors: Yehoshua SHEINMAN, Ra'anana (IL); Revital SHARIVKIN, Ramat-Gan (IL); Merav BELENKOVICH, Jerusalem (IL)

(73) Assignee: NOVELLUSDX LTD., Jerusalem (IL)

(21) Appl. No.: 15/536,582

(22) PCT Filed: Dec. 15, 2015
(86) PCT No.: PCT/IL2015/051218

§ 371 Date: Jun. 15, 2017

Related U.S. Application Data
(60) Provisional application No. 62/091,666, filed on Dec. 15, 2014.
(60) Provisional application No. 62/091,667, filed on Dec. 15, 2014.

(51) Int. Cl. C12N 15/10; B01J 19/00

(52) U.S. Cl. CPC C12N 15/1082; B01J 19/0046; B01J 2219/00659; B01J 2219/00623; B01J 2219/00596; B01J 2219/00497; B01J 2219/00376; B01J 2219/00743; B01J 2219/00619

(57) ABSTRACT

The present disclosure provides systems, kits, devices and methods for indirect transfer of multiple sets of nucleic-acid and other molecules to cells as exemplified by indirect transfection of sets of nucleic-acid molecules to viable cells.

### FIELD OF THE INVENTION

The present disclosure generally relates to indirect transfer of multiple sets of nucleic-acids and other molecules as exemplified by indirect transfection of sets of nucleic-acid molecules to viable cells.

### BACKGROUND OF THE INVENTION

Transferring multiple sets of various molecules to target viable cells is a valuable tool in the field of cellular biology. For example, transfection of nucleic-acid molecules to viable cells is an essential tool used to study and control gene expression. Nucleic-acid molecules can be transfected into cells to test multiple biochemical characterizations, the effects of gene expression on cell growth, gene regulatory elements and synthesis of proteins, among other nucleic-acid expression related fields.

Generally, various types of experiments require multiple sets of nucleic-acid molecules are transfected into different groups of cells for testing and studying in parallel the effects of their expression. A common method for parallel transfection of multiple sets of nucleic-acid molecules is carried out by utilizing plates having multiple wells, each containing viable cells, and deployed a different nucleic-acid molecule. This method is considered effective but not cost-effective for high-throughput tests, as the number of wells per plate is limited (commonly 12, 24, 48, 96, 384 or 1536 wells per plate) and the volume of reagents used is relatively high.

An attempt to miniaturize the transfection procedure was introduced with the reverse-transfection method, in which multiple sets of nucleic-acid molecules are deposited and dried on top a surface. Upon demand the printed surface is seeded with viable cells such that transfection takes place in different spots according to the location of the nucleic-acid sets. For example, U.S. Pat. No. 6,544,790 is directed to a reverse transfection method of introducing DNA of interest into cells and arrays, including microarrays, of reverse transfected cells. The reverse-transfection method optionally avails a higher density and increased number of transfection sites, compared to multi-well plates however, it is limited and disadvantageous in that: a) the deposited nucleic-acid molecules are not tethered to the surface and can therefore migrate upon contact with liquid or humidity, thus contaminating neighboring DNA spots. Consequently, the distinction/separation between different transfection sites is partial, high density of transfection sites is limited and the accuracy and reproducibility of the results may be impaired. b) The cells are not homogeneously seeded on the surface resulting in non-uniform cell density which may affect the transfection efficiency and introduce bias to the biological outcome.

There is thus a need in the art for systems, devices and methods for transferring multiple sets of molecules into cells that are high-throughput, low cost, time saving and which allow high transfer efficiency and homogeneity and that can further allow a more accurate comparison between different events.

### SUMMARY OF THE INVENTION

The present invention provides systems, devices, kits and methods for transferring of multiple sets of molecules into cells. In various embodiments, one or more of the above-described problems have been reduced or eliminated, while other embodiments are directed to other advantages or improvements.

According to some embodiments, there are provided systems, kits, devices and methods for depositing/transferring multiple sets of molecules in a predetermined array on a surface of a substrate bearing viable cells. In some embodiments, the systems, kits, devices and methods disclosed herein make use of a substrate bearing viable cells in a first predetermined array and a mesh capable of carrying multiple sets of the molecules arranged in a second predetermined array, such that the sets are separated from each other, wherein the mesh is configured to be approximated to the surface of the substrate and to release at least some of the molecules onto the surface of the substrate, while maintaining spatial separation of the sets within the second predetermined array. In some embodiments, the molecules are nucleic acids. In some embodiments, the first and second predetermined arrays spatially match. According to other embodiments the molecules are selected from the group consisting of peptides, proteins, antibodies, enzymes, lipids, metals and organic molecules or combinations thereof.

According to some exemplary embodiments, the systems, kits, devices and methods are utilized for transfecting nucleic-acid molecules into viable cells. In some embodiments, the systems, devices, kits and methods disclosed herein make use of a mesh to retain and release nucleic-acid molecules onto a substrate bearing or seeded with viable cells.

According to some embodiments, there is provided a system for depositing multiple sets of molecules in a predetermined array on a surface of a substrate bearing viable cells, the system comprising: a substrate bearing viable cells in a first predetermined array; a mesh configured to carry multiple sets of the molecules arranged in a second predetermined array, (which may optionally at least partially match the first array), such that said sets are separated from each other, wherein said mesh is configured to be approximated to the surface of the substrate and to release at least some of said molecules onto the surface of the substrate, while maintaining spatial separation of the sets within the second predetermined array; and a dispenser configured to dispense said multiple sets of molecules onto the mesh according to the second predetermined array. In some embodiments, the sets of molecules may be selected from the group consisting of: peptides, proteins, antibodies, nucleic acids, enzymes, lipids, metals and small organic molecules, or any combination thereof. Each possibility is a separate embodiment.

In some embodiments, the second predetermined array may be defined by a grid on said mesh. In some embodiments, the grid may be formed by a hydrophobic material deposited on the mesh. In some embodiments, the grid is non-toxic to the cells. In some embodiments, the grid may be applied by an automated applicator onto said mesh in a predetermined pattern, the predetermined pattern maintaining spatial separation between the sets of the second predetermined array. In some embodiments, the grid may be in fluid state prior to/while being applied and may solidify following its application.

According to some embodiments, the systems, devices, kits and methods disclosed herein allow an efficient, reliable, accurate, unbiased and cost effective transfection of nucleic acid molecules into target cells using low input volume and small unit area. According to some embodiments, there are provided systems, devices, kits and methods for providing transfection using a surface with multiple groups of viable cells disjointedly located on the surface and spatially separated by cell-free space between the different groups. Advantageously, providing spatial separation between the cell groups reduces/eliminates migration of transfected cells and/or nucleic-acid molecules, therefor providing better distinction between different transfection sites.

When used for cell transfection, the present methods, kits, devices and systems are surprisingly efficient and enable very accurate measuring with high comparability of results to standard transfection methods, as well as enabling high throughput at lower costs, according to some embodiments.

According to some embodiments, there is provided a system for transfection of nucleic acid molecules, the system includes a transparent substrate having a surface suitable for attachment of viable cells; a plurality of viable cells deposited on the surface in an array of predetermined locations that are not contiguous to one another; a mesh suitable for retention of nucleic-acid molecules (solutions containing thereof); and nucleic-acid molecules deposited in a designated array on said mesh, wherein said mesh is configured to be placed in alignment above the cells on said surface such that the interface between the mesh and the cells on said surface enables release of at least some of said nucleic-acid molecules, thereby transfecting at least some of said viable cells. In some embodiments, the nucleic acids are maintained in a water-based solution. In some embodiments, the nucleic acid molecules are deposited on the mesh in the presence of a transfection reagent.

In some embodiments, the nucleic-acid molecules comprise multiple nucleic-acid molecule sets arranged in the designated array on said mesh, such that said nucleic-acid molecule sets are separated from each other, wherein said mesh is configured to be placed above the cells on said surface and release at least some of said nucleic-acid molecule sets, thereby transfecting at least some of said viable cells, while maintaining spatial separation of the sets within the designated array. In some embodiments, the sets may be identical, similar or different from each other. In some embodiments, the nucleic acids are maintained in a water-based solution. In some embodiments, the nucleic acid molecules are deposited on the mesh in the presence of a transfection reagent.

According to some embodiments, the mesh may be made of any suitable material. In some embodiments, the mesh may be made of a polymeric material or combination of such materials. In some embodiments, the mesh may be made of a hydrophobic material. In some embodiments, the mesh may be made from a polymeric material having low wettability (for example, in the range of about 20-45 mN/m). In some embodiments, the mesh may be made from a hydrophobic polymer. In some embodiments, the mesh may made from such materials as, but not limited to: nylon, polyester, polyurethane, Polyethylene (PE), polyethylene terephthalate (PET), Polypropylene (PP), Polyvinyl chloride (PVC), Polytetrafluoroethylene (PTFE) mesh, Polyvinylidene fluoride (PVDF), Polydimethylsiloxane (PDMS), or combinations thereof. In some exemplary embodiments, the mesh is a nylon mesh. In some embodiments, the mesh may be made of a material which is optionally biologically, chemically and/or electrically inert to the retained solution or the solutions passing therethrough.

In some embodiments, the viable cells are homogeneously deposited within perimeters of the predetermined locations on said surface. In some embodiments, the viable cells are maintained in a suitable solution (such as, for example, cell culture medium).

In some embodiments, the substrate surface is suitable for attachment of a plurality of viable cell groups, wherein the viable cell groups are separated from each other, wherein locations of said viable cell groups correspond to locations of the nucleic-acid molecule sets such that upon approximating the mesh to the said cell groups on the surface, at least some nucleic-acid molecule sets are introduced to at least some corresponding viable cell groups, such that a specific nucleic-acid molecule set is introduced to a single viable cell group.

In some embodiments, the mesh includes a grid configured to define the separation between said nucleic-acid molecule sets within the designated array. In some embodiments, the grid is made of a hydrophobic material. In some embodiments, the hydrophobic material is non-toxic to the cells.

In some embodiments, the system may further include a nucleic-acid molecule dispenser configured to dispense the nucleic-acid molecule sets onto said mesh.

In some embodiments, the system may include a frame for maintaining the orientation of the mesh in alignment with the cells seeding surface. In some embodiments the system may include a frame for positioning/placing/stretching the mesh such that molecules deposited on it encounter an essentially flat uniform interface. In some embodiments the frame is further configured to enable separation between said mesh and said substrate, essentially without affecting the cells. In some embodiments, the system may include a framed container configured to facilitate detaching of the mesh from the substrate essentially without affecting the cells. In some embodiments, the system may include a casing (framed container), configured to hold the substrate and said mesh in alignment and to further allow subsequent incubation steps of the substrate with or without the mesh, optionally immersed in suitable fluid. In some embodiments, the casing may further provide a float structure to enable separation between said mesh and said substrate, essentially without affecting the cells. In some embodiments, the framed container may include a flotation means configured to facilitate the detachment of the seeding mesh from the substrate. In some embodiments, a float device/element, if used, may be attached or otherwise be associated with the mesh frame. In some embodiments, the frames may be separate frames or one frame configured to enable one or more of the above mentioned configurations.

According to some embodiments, there is provided a kit for transfection of nucleic acid molecules, the kit comprising: a mesh deposited with nucleic-acid molecules sets arranged in a designated array defined by a grid on said mesh, wherein the nucleic-acid molecules sets are separated from each other by the grid, wherein the grid maintains spatial separation of the sets within the designated array; wherein the mesh is configured to be placed above a substrate surface bearing viable cells arranged in a predetermined pattern and to release at least some of the nucleic-acid molecules to be transfected to at least some of the viable cells, while maintaining spatial separation of the sets within the designated array, wherein the designated array aligns with at least part of the pattern of the target cells.

In some embodiments, the kit may further include a substrate (optionally transparent) having a surface configured for attachment of viable cells.

In some embodiments, the kit may further include a frame for maintaining the orientation of the mesh in alignment with the substrate surface bearing the viable cells.

In some embodiments, the kit may further include transfection agents deposited with the nucleic-acid molecules.

In some embodiments, the grid is hydrophobic and non-toxic to the cells.

In some embodiments, the frame of the kit is further configured to enable detachment of said mesh from the substrate surface, essentially without effecting the cells.

According to some embodiments, there is provided a method for transfecting nucleic-acid molecules into cells, the method comprising: providing a mesh comprising nucleic-acid molecules deposited on the mesh in a designated array; providing a substrate comprising a surface deposited with viable cells (optionally at a controlled seeding density) in an array of predetermined locations; and approximating a surface of the mesh to the viable cells on the substrate surface such that at least some nucleic-acid molecules are introduced to at least some of the viable cells, thereby transfecting at least some of the viable cells.

In some embodiments, the nucleic-acid molecules may include a plurality of nucleic-acid molecule sets deposited on said mesh in a designated array, such that said nucleic-acid molecule sets are separated from each other, wherein upon approximating the surface of the mesh to the cells on the substrate surface at least some nucleic-acid molecule sets are transfected into at least some of the viable cells, while maintaining spatial separation of the sets within the designated array. In some embodiments, the mesh may include a grid that maintains spatial separation between the nucleic acid molecule sets within the designated array.

In some embodiments, the nucleic-acid molecules may be deposited on the mesh in the presence of a transfection reagent. In some embodiments, approximating the surface of the mesh to the viable cells on the substrate surface may be performed in aqueous solution. In some embodiments, the viable cells are maintained in a suitable solution. In some embodiments, the viable cells may be divided to a plurality of groups, wherein the groups are separated from each other. In further embodiments, the viable cells are divided to a plurality of groups, wherein said groups are separated from each other, wherein locations of said viable cell groups correspond to locations of said nucleic-acid molecule sets, such that upon approximating said mesh to said cells on the surface, at least some nucleic-acid molecule sets are transfected to at least some corresponding viable cell groups, such that a specific nucleic-acid molecule set is introduced to a specific viable cell group.

In some embodiments, the method may further include dispensing the plurality of nucleic-acid molecule sets on the mesh according to the designated array. In further embodiments, the method may further include incubating the mesh with the viable cells on the substrate; and separating the mesh from the cells on the substrate, (essentially without affecting the cells), thereby obtaining a substrate with multiple sets of transfected cells. In some embodiments, the substrate is transparent.

According to some embodiments, there is provided a system for transfection of nucleic acid molecules, the system includes a cell-slide having a surface configured to carry viable cells, a plurality of viable cells deposited on the surface, a mesh configured to carry nucleic-acid molecules and multiple nucleic-acid molecule sets located at predetermined locations on the mesh, wherein the mesh is configured to be placed on the surface and release/deploy at least some of the nucleic-acid molecule sets onto the surface to be transfected to at least some of the viable cells. In some embodiments, the mesh includes a grid configured to determine the locations of the nucleic-acid molecule sets, so as to form an array. In some embodiments, the grid is made of a solid or semi-solid, hydrophobic, non-toxic material. According to some embodiments the system further includes a nucleic-acid molecules dispenser/printer configured to deploy nucleic-acid molecules onto the mesh. In some embodiments, the dispenser may be manual, automatic or semi-automatic. In some embodiments, the system may further include a frame, configured to allow positioning/alignment of the mesh carrying the nucleic acid molecules and the substrate carrying the cells. In some embodiments, the system may include a container, configured to hold the slide and/or the mesh and to allow subsequent incubation steps of the substrate with or without the mesh, optionally immersed in suitable fluid and/or enable a floating of the mesh to facilitated separation between the mesh and the slide, essentially without affecting the cells.

According to some embodiments there is provided a device for transfection of nucleic acid molecules, the device includes: a cell-slide having a surface configured to carry viable cells and a mesh configured to carry nucleic-acid molecules located at predetermined locations on the mesh, wherein the mesh is configured to be placed on the surface and release/deploy at least some of the nucleic-acid molecules on the surface to be transfected to at least some of the viable cells.

According to some embodiments there is provided a method for transfecting nucleic-acid molecules into cells, the method includes the steps of: providing a mesh including a plurality of nucleic-acid molecule sets and/or transfection mixes absorbed and/or retained within the mesh, providing a substrate with a cell seeding surface, having viable cells attached to the seeding surface; and approximating the mesh to the cells such that at least some nucleic-acid molecule sets are introduced to the viable cells.

According to some embodiments, the mesh is configured to reversibly absorb and/or retain nucleic-acid molecules (in a water-based solution, which may include transfection agents). In some embodiments, the method may further include: incubating the substrate with the mesh and separating the mesh from the substrate, thereby obtaining a substrate with a multiple transfected cells or cell groups.

According to some embodiments, there is provided a system for depositing multiple sets of molecules in a predetermined array on a surface of a substrate bearing viable cells, the system comprising: a substrate bearing viable cells in a first predetermined array; a mesh configured to carry multiple sets of the molecules arranged in a second predetermined array, such that said sets are separated from each other, wherein said mesh is configured to be approximated to the surface of the substrate and to release at least some of said molecules onto the surface of the substrate, while maintaining spatial separation of the sets within the second predetermined array; and a dispenser configured to dispense said multiple sets of molecules onto the mesh according to the second predetermined array.

According to some embodiments, the sets of molecules are selected from the group consisting of: peptides, proteins, antibodies, enzymes, polymers, nucleic acids, metals, lipids and small organic molecules. Each possibility is a separate embodiment.

According to some embodiments, the second predetermined array is defined by a grid on said mesh. According to some embodiments, the grid is formed by a hydrophobic material deposited on the mesh. In some embodiments, the hydrophobic material is non-toxic to the cells. According to some embodiments, the grid may be applied by an automated applicator onto said mesh in a predetermined pattern, said predetermined pattern maintaining spatial separation between the sets of the second predetermined array. In some embodiments, the system may further include a frame, configured to facilitate alignment of said substrate and the mesh. In some embodiments, the frame is configured to enable separation between said mesh and said substrate, essentially without affecting the cells on the substrate. In some embodiments, the substrate is transparent.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions. The following embodiments and aspects thereof are described and illustrated in conjunction with systems, kits, devices and methods which are meant to be exemplary and illustrative, not limiting in scope.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIGS. 1A-C schematically illustrate a system for an indirect transfer of molecules to viable cells, according to some embodiments; FIG. 1A schematic illustration of a substrate bearing cells and a mesh having multiple sets of molecules, arranged in an array, dictated by a grid printed on the mesh; FIG. 1B schematic illustration of the substrate bearing cells and the mesh when the mesh is approximated to the substrate;

FIG. 1C schematic illustration of a substrate bearing cells after transfer of the molecules from the mesh to the cells on the substrate;

FIG. 2A shows an illustration of a perspective view of a casing configured to hold a substrate having a surface suitable for attachment of viable cells according to some embodiments;

FIG. 2B shows an illustration of a top perspective view of a mesh-holding frame configured to hold/stretch a mesh, according to some embodiments;

FIG. 2C shows an illustration of a top view of a substrate casing (holding a substrate) and a mesh-holding frame (holding a mesh), the mesh frame being positioned on top of the substrate according to some embodiments;

FIGS. 2D-E show illustrations of cross section views of a float including a substrate and a mesh, according to some embodiments; FIG. 2D—The mesh is in contact with the substrate; FIG. 2E—the mesh is separated from the substrate;

FIG. 2F shows a schematic illustration of a top view of a substrate casing with substrate and a mesh-holding frame (holding a mesh) associated with a float device, the mesh positioned on top of the substrate, according to some embodiments;

FIG. 2G shows a schematic illustration of a cross section view of a substrate casing and a mesh-holding frame (holding a mesh), the mesh frame being associated with a float device, according to some embodiments;

FIG. 3 shows a schematic illustration of a cell seeding kit, according to some embodiments;

FIG. 4A shows a schematic illustration of molecules dispenser (printer), according to some embodiments;

FIG. 4B shows a schematic illustration of a mesh, prior to being gridded, according to some embodiments;

FIG. 4C shows a schematic illustration of a mesh including a grid, forming an array of grid-free chambers, according to some embodiments;

FIG. 4D shows a schematic illustration of a mesh with multiple molecules sets printed thereon according to some embodiments;

FIG. 4E shows a perspective front view illustration of an exemplary solution dispenser (printer), according to some embodiments;

FIG. 5A a pictogram showing an example of cells seeded on a substrate, through 100 μm² pores of a polymeric mesh, thus assuming the mesh's weaving pattern;

FIG. 5B a pictogram of a mesh gridded with hydrophobic polymer lines to form an array of 2 mm×2 mm chambers;

FIG. 5C a pictogram of cell groups seeded in 2 mm² spot pattern on a substrate using the mesh of FIG. 5B.

FIGS. 6A-C pictograms of cell groups seeded on a substrate surface through a polymeric mesh, spatially separated according to a pattern dictated by the mesh's grid; the pattern is maintained after the mesh is removed/eliminated from the FIG. 6A is the result of manual seeding; FIG. 6B-C are the result of automated-based seeding. Cell nuclei were labeled with DAPI and the image was acquired under UV lighting, 10× magnification; FIG. 6A 20×20 image stitching; FIG. 6B-C 8×15 image stitching.

FIGS. 7A-C pictograms showing examples of various fluorescent protein expression in cells which is the result of different nucleic-acid molecules manual transfection to cells seeded on a substrate, according to some embodiments; and

FIGS. 7D-E images of cells in a single spot (chamber) on the array seeded and transfected using automated tooling. FIG. 7D shows fluorescent cells, which are cells that underwent transfection and express KRAS and ERK2-GFP. FIG. 7E shows DAPI staining of the nucleic of the same cells. The images are of 10× magnification; 4×4 image stitching.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, the present disclosure provides methods, systems, kits and devices that bring advantageous features for transfer of molecules, to a substrate, for example, into viable cells. The molecules may include any type of molecules capable of being deployed to the cells, and may include such molecules as, but not limited to: nucleic acid molecules, enzymes, lipids, metals, proteins, small organic molecules, beads, and the like or combinations thereof. Some of the features include potentially high-throughput and low cost transferring of such molecules for performing large numbers of different assays simultaneously at lower input volumes and a less space consuming platform. Other features include high accuracy of the assays resulting from spatial separation between different molecules to be deposited, which may be arranged in an addressable array, and the unified homogeneous density of seeded cells across the surface on which the cells are deployed.

According to some exemplary embodiments, the present disclosure provides methods, systems, kits and devices that bring advantageous features for transfection of nucleic-acid molecules into viable cells. Such features include, high-throughput and low cost transfection for performing large numbers of different transfections simultaneously, significantly lower input volumes, smaller space consumption, a very accurate and effective transfection as well as increased throughput of downstream assays attributed to the smaller platform surface, the spatial separation between different cell groups arranged in an addressable array and the unified homogeneous concentration of the seeded cells.

The following are terms which are used throughout the description and which should be understood in accordance with the various embodiments to mean as follows:

The term “nucleic-acid molecule” as used herein also may refer to a nucleic-acid of known sequence or source, a nucleic-acid of interest or a nucleic-acid to be introduced into cells. As referred to herein, the terms “nucleic-acid”, “nucleic-acid molecules” “oligonucleotide”, “polynucleotide”, and “nucleotide” may interchangeably be used herein. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA or RNA molecules may be, for example, but not limited to: complementary DNA (cDNA), genomic DNA, synthesized DNA, recombinant DNA, or a hybrid thereof or an RNA molecule such as, for example, mRNA, shRNA, siRNA, miRNA, Antisense RNA, and the like. Each possibility is a separate embodiment. The terms further include oligonucleotides composed of naturally occurring bases, sugars, and covalent inter-nucleoside linkages, as well as oligonucleotides having non-naturally occurring portions, which function similarly to respective naturally occurring portions. In some embodiments, the nucleic acid molecules may be conjugated to other molecules, such as fluorescent proteins or glycosylated/phosphorylated groups. The term nucleic acid molecules encompass “nucleic acid construct” and “expression vector”. In some embodiments, nucleic acid molecules may be provided as is or in a suitable solution/fluid/medium.

The terms “nucleic acid construct” and “construct” may interchangeably be used. The terms refer to an artificially assembled or isolated nucleic-acid molecule which may include one or more nucleic-acid sequences, wherein the nucleic-acid sequences may include coding sequences (that is, sequence which encodes an end product), regulatory sequences, non-coding sequences, or any combination thereof. The term “Expression vector” refers to constructs that have the ability to incorporate and express heterologous nucleic-acid fragments (such as, for example, DNA), in a foreign cell.

The term “expression”, as used herein, refers to the production of a desired end-product molecule in a target cell. The end-product molecule may include, for example an RNA molecule; a peptide or a protein; and the like; or combinations thereof. In some the expression may be identified by identifying the end product in the cell, for example, by biochemical methods, analytical methods, imaging methods, and the like.

As used herein, the terms “introducing” and “transfection” may interchangeably be used and refer to the transfer of molecules, such as, for example, nucleic-acids, polynucleotide molecules, vectors, and the like into a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, CRISPR and the like, or combinations thereof. The transfection of the cell may be performed on any type of cell, of any origin, such as, for example, human cells, animal cells, plant cells, viruses, and the like. The cells may be selected from isolated cells, tissue cultured cells, cell lines, primary cultures, cells obtained from an organism body, cells obtained from a biological sample, and the like.

The terms “polypeptide,” “peptide” and “protein” are used interchangeably herein to refer to a polymer of amino acid residues. The terms apply to naturally occurring amino acid polymers, to amino acid polymers in which one or more amino acid residue is an artificial chemical analogue of a corresponding naturally occurring amino acid, as well as to amino acid polymers having one or more tags or any other modification. Specific examples of proteins include antibodies, enzymes and some types of antigens.

The term “bead(s)” refer to any type of bead that can be used in biological applications. In some embodiments, the bead may have a globular shape. In some embodiments, the beads may range in size from nanometric to micrometric size. In some embodiments, the beads may be made of any suitable material. In some embodiments, the beads may be coated with one or more materials, compounds or molecules. In some embodiments, the beads are inert. In some embodiments, the beads are chemically, biologically and/or electrically inert. In some embodiments, the beads are glass beads, metal beads, polymeric beads, magnetic beads, and the like.

As used herein, the terms “substrate”, “slide”, “cell slide” and “transfection slide” may interchangeably be used. The terms are directed to a solid or semi-solid substrate onto which cells may be seeded, deployed, dispensed, dispersed, attached, adhered, tethered, placed, grown, and the like, and/or to which molecules are being transferred from the mesh. In some embodiments, the cells carried by the substrate may be transfected or may be deposited by other molecules. In some embodiments, the substrate may have any regular or irregular shape, such as, rectangular, circular, elliptical, and the like. In some embodiments, the substrate may have a substantially flat planar surface. In some embodiments, the substrate may be transparent. The substrate may be made of such materials as, glass, quartz, plastic, polystyrene, polypropylene, various types of gels, and others. In some embodiments, the substrate may be coated with various materials (as detailed below). In some embodiments, the coating may be on the surface configured to carry the cells. In some embodiments, the coating may be on more than one surface of the slide.

According to some embodiments, the substrate may be made of a solid, rigid or semi-rigid material designed to withstand stress and strain forces and/or withstand various temperatures. In some embodiments, the properties of the substrate are selected to match the assay in which it is used. In some embodiments, the substrate is transparent. In some embodiments, the substrate is opaque.

According to some embodiments, the substrate has a rectangular surface having a length in the range of about 2-30 cm. In some embodiments, the substrate has a rectangular surface having a length in the range of about 2-20 cm. In some embodiments, the substrate has a rectangular surface having a length in the range of about 7-15 cm. In some exemplary embodiments, the substrate has a rectangular surface having a length of approximately 7.5 cm. In some embodiments, the substrate has a width in the range of about 1-30 cm. In some embodiments, the substrate has a width in the range of about 5-20 cm. In some embodiments, the substrate has a width of approximately 2.5 cm. In some embodiments, the substrate has a depth in the range of about 0.01-1 cm. In some embodiments, the substrate has a depth in the range of about 0.05-0.5 cm. In some embodiments, the substrate has a depth of about 0.1-0.15 cm (for example, 0.11 cm). According to some embodiments, the substrate has a rectangular surface having a length to width ratio in the range of about 1-10. According to some embodiments, the substrate has a rectangular surface having a length to width ratio in the range of about 2-5. According to some embodiments, the substrate has a rectangular surface having a length to width ratio of approximately 3. According to some embodiments, the substrate has a circular surface. In some embodiments, the substrate has a surface area of about 18.75 cm². In some embodiments, the substrate has a surface area in the range of about 1-500 cm².

The term “cells” as used herein may refer to any cell, mammalian and non-mammalian cells, Eukaryotic and Prokaryotic cells or any other type of cells of interest. Exemplary cells can include, for example, but not limited to, of mammalian, avian, insect, yeast, filamentous fungi or plant origin. Non-limiting examples of mammalian cells include human, bovine, ovine, porcine, murine, and rabbit cells. The cell may be a primary cell or a cell line. In some embodiments, the cells may be selected from isolated cells, tissue cultured cells, cell lines, primary cultures, cells obtained from an organism body, cells obtained from a biological sample, and the like. In some embodiments, the cells may be selected from HeLa cells, HEK 293 cells, PC12 cells, U2OS cells NCI60 cell lines (such as, A549, EKVX, T47D, HT29), and the like or combination thereof. Each possibility is a separate embodiment. In some embodiments, the cells are other than osteoprogenitor cells. In some embodiments, the cells may be manipulated cells. In some embodiments, the manipulated cells are transfected with an exogenous gene. In some embodiments, the manipulated cells transiently or stably express one or more exogenous genes. In some embodiments, the cells are viable, living cells. In some embodiments, the term “cell” may further encompass cells in a medium (such as, growth medium), fluid, solution, buffer, serum or other bodily fluids. In some embodiments, the term “seeding” is directed to placing, deploying, dispensing, attaching, adhering, tethering, placing, growing cells on a substrate. In some embodiments, the cells may be used for various applications and assays prior to or after molecules have been transferred thereto. For example, the cells may be used in biochemical assays (such as, for example, but not limited to: immunostaining, enzymatic reactions, and the like), molecular biology assays (such as, for example, but not limited to: PCR); imaging assays (such as, but not limited to: microscopy (such as, fluorescent microscopy, confocal microscopy, and the like), and the like.

The term “cell group(s)” as used herein may refer to a plurality of cells deployed on a surface of a slide in relatively close approximation. In some embodiments, a cell group is spatially separated from other cell groups. According to some embodiments a “cell group” may occupy a certain space or a spot or a chamber or a location or on the surface of the substrate. In some embodiments, cell groups are arranged in an array/matrix. In some embodiments, the array may be predetermined. In some embodiments, the array may be an addressable array. In some embodiments, the array may be a designated array.

According to some embodiments, the number of cells per cell group is more than about 1*10² cells. In some embodiments, the number of cells per cell group is less than about 5*10⁵ cells.

According to some embodiments, cell density in cell groups is more than about 1*10³ cells/cm² and less than about 2*10⁵ cells/cm².

According to some embodiments, viable cells may include any type of cell, such as, human cell, animal cell, avian cell, plant cell and the like. In some embodiment, the viable cells are adherent cells. In some embodiments, the cells are tissue culture cells. In some embodiments, the cells are tissue-derived cells. In some embodiments, the cells are from a cell line.

As used herein, the term “mesh” refer to a porous structure having multiple pores/apertures configured to allow controllable passage and/or retaining of liquid/cells/molecules through/within the pores/apertures. A mesh may be a film made of a network of wires, strands or threads, attached, woven or interlaced to form multiple apertures. According to some embodiments, the apertures of the mesh have a predetermined density and properties depending on the matter to be passed through and/or retained within the apertures, or according to the properties of the desired outcome/pattern. According to some embodiments, the mesh may have any desired pattern/structure. According to some embodiments, the mesh may be extruded, oriented, expanded, woven or tubular; the mesh may be made from connected (for example, woven) strands of polymer(s) (such as inert materials) that define a mesh structure with a mesh pattern confining the plurality of holes/apertures in the mesh. According to some embodiments, the mesh may have a weaving pattern confining the holes thereof. According to some embodiments, the mesh may have a lattice structure confining the holes thereof. According to some embodiments, a mesh may be a web, a net, a lattice, a honeycomb, a matrix, and the like. According to some embodiments, the mesh may be made of a polymeric material. In some embodiments, the mesh may be made from a polymeric hydrophobic material. In some embodiments, the mesh may be made from a polymeric material having low wettability. In some embodiments, low wettability may be in the range of about 20-45 mN/m, and any subranges thereof. As referred to herein, the term “wettability” refers to the ability of a solid surface to reduce the surface tension of a liquid. The term “wetting” as used herein refers to the ability of a liquid to maintain contact with the solid surface.

According to some embodiments, the mesh and pores/apertures thereof are configured such that capillary forces are introduced when the mesh is introduced with fluid (for example, water-based solutions) or when the mesh comes in contact with a wet surface. According to some embodiments, the mesh and pores/apertures thereof are configured such that capillary forces are introduced when the mesh is printed with liquid solutions or when the mesh is placed on a wet substrate.

According to some embodiments, a mesh may be configured to controllably avail/allow passage of viable cells through the apertures thereof. A mesh according to the preceding configuration may be termed herein “cell-mesh”, “cell-sheet”, “cell-seeding sheet”, “cell-seeding mesh” and/or “seeding mesh”. According to some embodiments, a cell-mesh may be configured to have apertures having a size in the range of about 20-500 μm. According to some embodiments, a cell-mesh may be configured to have apertures having a size in the range of about 50-350 μm. According to some exemplary embodiments, a cell-mesh may be configured to have apertures of approximately 100 μm in size. According to some embodiments, a cell-mesh may be configured to have apertures of any appropriate size. According to some embodiments, a mesh may have any density/concentration of apertures per area unit.

According to some embodiments, a mesh may be configured to controllably retain solutions containing molecules (such as, for example, nucleic-acid molecules), within the apertures of the mesh. According to some embodiments, a mesh may be configured to controllably avail/allow passage of molecules and/or solution containing them through the apertures of the mesh. In some embodiments, such mesh may be termed herein “mesh”, “molecules mesh” and/or “printing mesh”. In some exemplary embodiments, the molecules are nucleic acid molecules. A mesh according to the preceding configuration may be termed herein “mesh”, “nucleic-acid-mesh”, “DNA mesh”, and/or “nucleic-acid molecule printing mesh”. According to some embodiments, a printing mesh is configured to have apertures in the range of about 5-200 μm. According to some embodiments, a printing mesh is configured to have apertures in the range of about 10-100 μm. In some exemplary embodiments, a printing mesh is configured to have apertures of approximately 41 μm. According to some embodiments, a printing mesh is configured to have apertures of more than 10 μm.

According to some embodiments, a ratio between aperture size in a seeding mesh and/or aperture size in a printing mesh may be in the range of about 1:1-20:1. According to some embodiments, a ratio between aperture size in a seeding mesh and/or aperture size in a printing mesh may be in the range of about 1:1-10:1. According to some embodiments, a ratio between aperture size in a seeding mesh and/or aperture size in a printing mesh may be approximately 2.5:1.

According to some embodiments, a mesh may be made of any suitable material. In some embodiments, the mesh may be made of a polymeric material or combination of such materials. In some embodiments, the mesh may be made from a polymeric material having low wettability (for example, in the range of about 20-45 mN/m). In some embodiments, the mesh may be made from a hydrophobic polymer. For example, a mesh may be made from such materials as, but not limited to: nylon, polyester, polyurethane, Polyethylene (PE) polyethylene terephthalate (PET), Polypropylene (PP), Polyvinyl chloride (PVC), Polytetrafluoroethylene (PTFE), Polyvinylidene fluoride (PVDF), Polydimethylsiloxane (PDMS), glass, and the like, or combinations thereof. Each possibility is a separate embodiment.

In some embodiments, the mesh is a polymeric material. In some embodiments, the mesh is a polymeric material having low wettability. In some embodiments, the mesh is a film made or comprised of multiple connected or woven strands of flexible/ductile materials in a pattern generating open spaces between strands. In some embodiments, the mesh may be made of a material which is optionally biologically, chemically and/or electrically inert to the retained solution. In some exemplary embodiments, the mesh may be selected from, but not limited to: a nylon mesh, polyester mesh, polyurethane mesh, Polyethylene (PE) mesh, polyethylene terephthalate (PET) mesh, Polypropylene (PP) mesh, Polyvinyl chloride (PVC) mesh, Polytetrafluoroethylene (PTFE) mesh, Polyvinylidene fluoride (PVDF) mesh, Polydimethylsiloxane (PDMS) mesh, glass mesh, and the like. Each possibility is a separate embodiment.

According to some embodiments, a mesh may be made of any suitable material. In some embodiments, the mesh may be made of a polymeric material or combination of such materials. In some embodiments, the mesh may be made of an hydrophobic material. In some embodiments, the mesh may be made from a polymeric material having low wettability (for example, in the range of about 20-45 mN/m). In some embodiments, the mesh may be made from a hydrophobic polymer. For example, a mesh may be made from such materials as, but not limited to: nylon, polyester, polyurethane, Polyethylene (PE), polyethylene terephthalate (PET), Polypropylene (PP), Polyvinyl chloride (PVC), Polytetrafluoroethylene (PTFE) mesh, Polyvinylidene fluoride (PVDF), Polydimethylsiloxane (PDMS), glass, or the like.

As used herein, the terms “restrainer”, “restraining grid”, “grid”, “cell restrainer”, “DNA restrainer” and/or “nucleic-acid molecule restrainer” refer to a material configured to be placed/mounted on, soaked, at least partially or completely within, or integrated in a mesh and to obstruct passage of liquids, cells, beads, molecules (such as nucleic-acid molecules) or solutions containing such molecules, in the mesh pores in which it is placed, soaked and/or integrated. According to some exemplary embodiments, the grid is configured to repel hydrophilic and/or water-based solutions, such as, for example, cell-containing solutions or nucleic-acid molecules-containing solutions from the region in which it is placed, soaked and/or integrated. According to some embodiments, the grid is shaped to provide “restrainer-free” areas confined by the restrainer; the restrainer-free areas are configured to allow passage of cells, cell-containing solutions, beads, beads-containing solutions, molecules, and/or molecules-containing solutions. In some embodiments, the “restrainer-free” areas form a matrix/array.

According to some embodiments, the restrainer is shaped to form a network of lines that cross each other to form a series of squares or rectangles, or any desired form. According to some embodiments, the grid creates a matrix/array of regions/spots/small chambers/compartments/elements confined by the lines of the grid. In some embodiments, the grid may form an addressable array. According to some embodiments, the grid lines have predetermined thickness (line width and/or height) and spaced apart by a certain predetermined spacing areas according to the desired shape and area of the chambers/compartments/elements of the array. In some embodiments, the grid defines the perimeters of the chambers and/or the array.

According to some embodiments, the grid may be made of a liquid or semi-liquid hydrophobic material, capable of solidifying, which may be a thermoplastic polymer, thermally cured, liquid soluble polymer, a photo-initiated polymer, non-toxic hydrophobic material, and the like. In some embodiments, the grid is hydrophobic. In some embodiments, the grid is firm after solidifying. In some embodiments, the grid material is non-toxic to the cells.

According to some embodiments, grid lines of a cell-mesh grid may be wider than grid lines of a molecules-mesh grid.

The terms “Array” and “matrix” as used herein refers to the arrangement of objects on a surface, so as to form an arrangement of separated chambers/compartments/elements. In some embodiments, the array is systematic. In some embodiments, the array may be formed by cross lines (for example, horizontal lines, vertical lines, diagonal lines, circular lines, and the like). In some embodiments, the array is arranged in the form of columns and rows. In some embodiments, the cross lines may be physical lines, or virtual lines, providing separation between the various elements/chambers/compartments of the array. In some embodiments, the array is an “addressable array” (also referred to as a “designated array”), that is, the location of the various chambers are identifiable and recognizable and each may be assigned an “address” which is indicative of its relative location within the array. In some embodiments, the shape, size, distribution and/or dimension of the compartments/chambers forming the array may be predetermined.

As used herein, the terms “float” or “float device” refer to a device configured for facilitate removal of a mesh (gridded or not) off the slide following seeding of the slide, incubating the slide with a mesh and/or following performing transfection on the slide. The float is designed to carry a frame holding the cell-mesh, with or without aligned transparency, or to carry a frame holding a molecules mesh and align it over the substrate for the duration of incubation of the relevant mesh with the substrate. The float device is equipped with floating elements allowing detachment of the mesh carried by the device from the substrate at the end of the incubation period without pilling cells off the slide. The float device is attached, connected, or otherwise associated with the mesh-holding frame which is placed in close proximity over a dedicated substrate to allow precise alignment of the mesh and substrate.

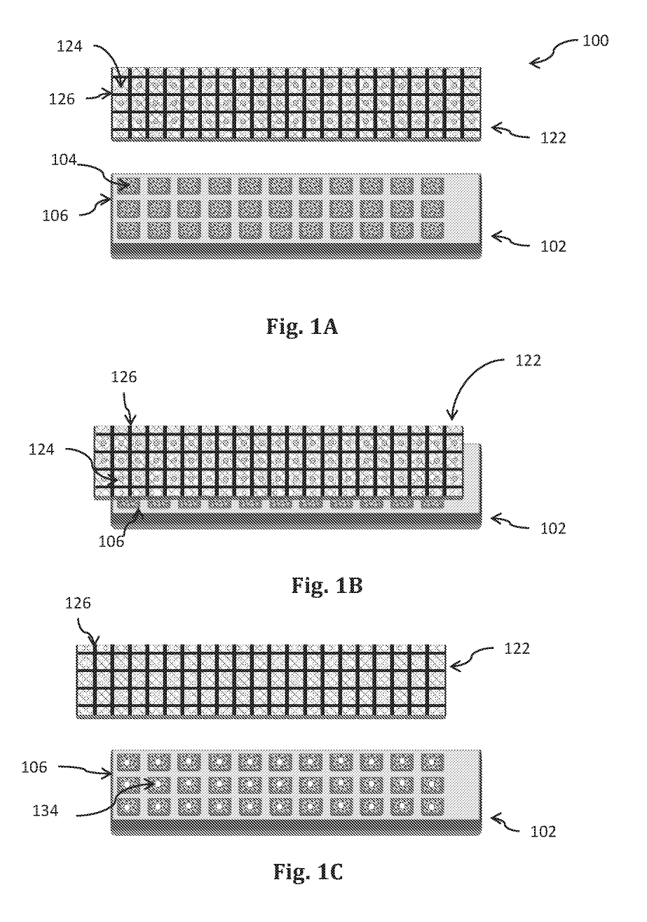

Reference is now made to FIGS. 1A-C, which schematically illustrate a system, 100, for indirect transfer of nucleic acid and other molecules into cells, according to some embodiments. In FIG. 1A, a substrate 102 is shown, which includes multiple groups of cells 104 separated by cell-free area 106 (i.e. the cells may be arranged in an array of predetermined locations that are not contiguous to one another). Additionally, a mesh 122 is shown having multiple molecule sets 124 (for example, nucleic acid molecules), separated by a grid 126, in a designated array.

According to some embodiments, substrate 102 may comprise a coated surface configured to carry viable cells (i.e. suitable for attachment of viable cells). In some embodiments, the surface may be coated or formed with various materials configured to improve cell adherence/attachment to the substrate. A suitable coating may be, for example, but not limited to: Poly-l-lysine, poly-D-lysine, Aminosilanes, Poly-l-ornithine, Collagen, Fibronectin, Laminin, or any combination thereof. Each possibility is a separate embodiment.

According to some embodiments, cell groups 104 and molecule sets 124 are located on substrate 102 and mesh 122, respectively, in matching locations/positions, such that upon approximating mesh 122 to slide 102 at least some molecule sets are transferred/introduced to matching cell groups 104. When mesh 122 is placed in alignment above cells 104, the interface between the mesh and the cells on the surface enables release of at least some of said sets of molecules, thereby transferring at least some of the molecules sets to the cells, maintaining spatial separation of the sets within the designated array.

FIG. 1B illustrates mesh 122 being approximated to substrate 102. According to some embodiments, capillary forces may fasten/connect mesh 122 to substrate 102. Capillary forces may serve to facilitate movement/transfer of molecule sets 124 (such as, for example, nucleic acid molecules) to cell groups 104, to further allow/enable interaction (such as, transfection of the nucleic acid molecules) with the target cells. According to some embodiments, cell-free area 106 on the substrate is a grid-shaped area (array) having horizontal lines and vertical lines with a certain predetermined width, and grid 126 has horizontal lines and vertical lines. According to some embodiments, the horizontal lines and vertical lines of cell-free area 106 are of larger width than vertical lines and horizontal lines of grid 126. Advantageously, upon approximating mesh 122 to slide 102 at least some vertical lines and horizontal lines of grid 126 fall completely within the horizontal lines and vertical lines of cell-free area 106 such that grid 126 or parts of grid 126 do not come in contact with cell groups 104. Such a setting ensures that the cells are not harmed nor affected by approximating or incubating the mesh carrying the molecules (and/or the grid on said mesh) with the cells.

FIG. 1C illustrates mesh 122 being separated from substrate 102. After transferring molecule sets 124 to cell groups 104, interaction (such as transfection in an exemplary case of nucleic acid molecules) may take place. Multiple parallel interaction (such as transfection) sites 134 may result from the above described transfer, while cell-free area 106 provides spatial separation between the different interaction sites (134).

According to some exemplary embodiments, grid 126 may be made of a non-toxic hydrophobic polymer arranged in perpendicular or semi-perpendicular lines having a width of about 0.5-1.5 mm and density of about 2-9 (for example, 2.9) horizontal lines per cm and 2-9 (for example, 2.9) vertical lines per cm, resulting in nucleic-acid hosting chambers with a surface area of 0.9-9 mm².

According to some embodiments, cell-free area 106 has perpendicular or semi-perpendicular horizontal lines and vertical lines.

According to some exemplary embodiments, mesh 122 is made of a polymeric material having low wettability which may be selected based on the molecules that will interact/transfer therethrough. In some exemplary embodiments the mesh is a nylon mesh.

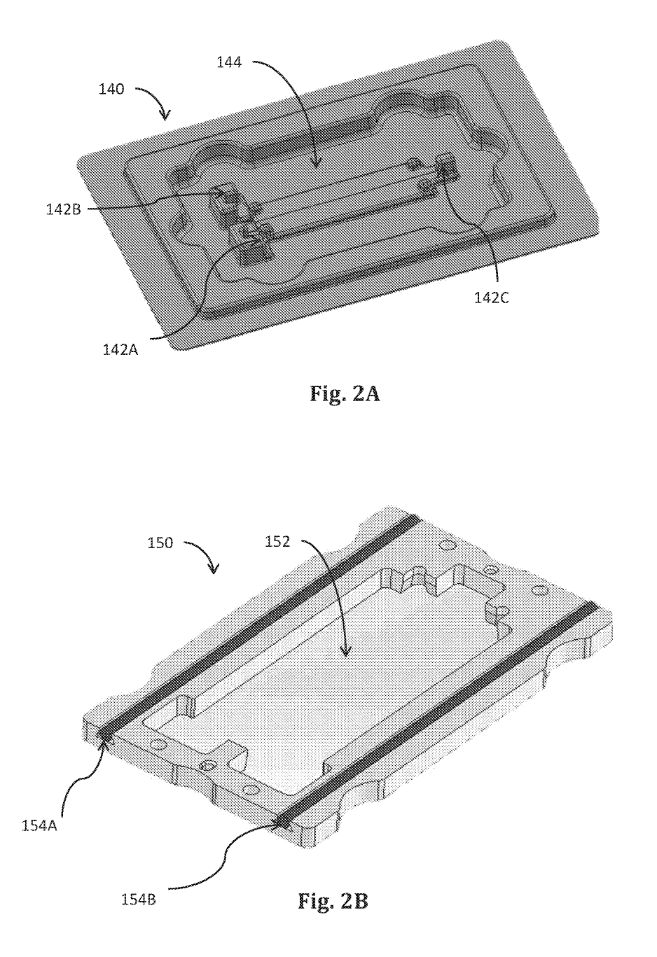

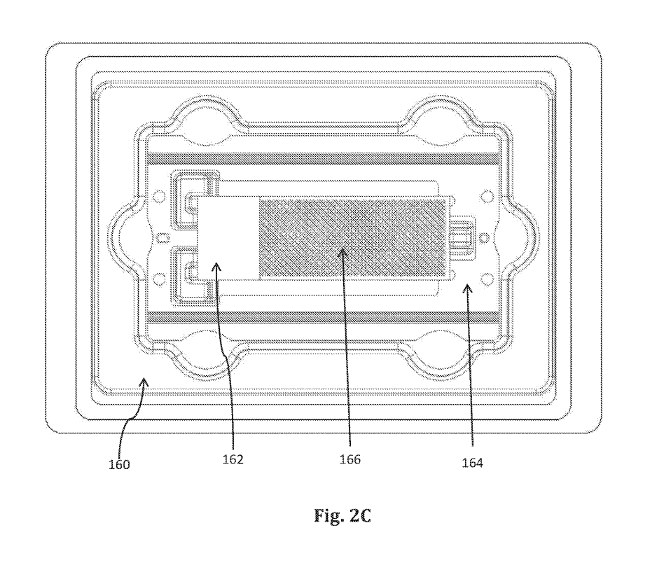

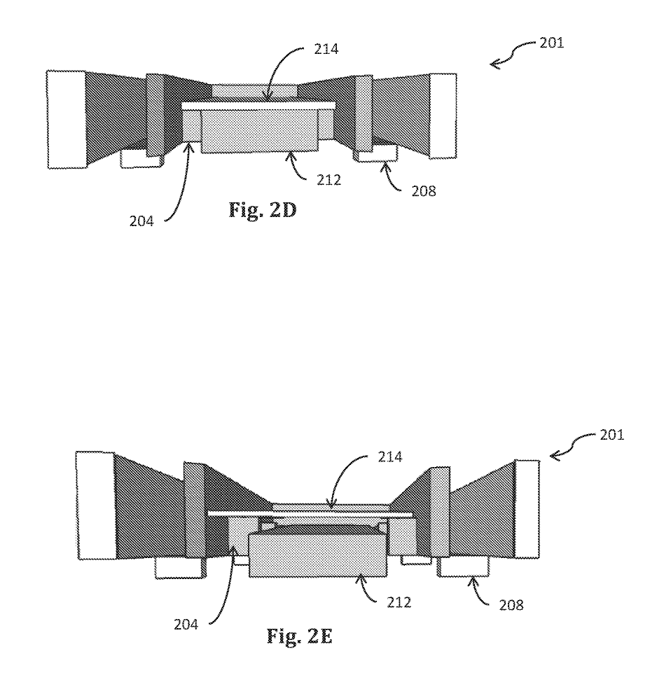

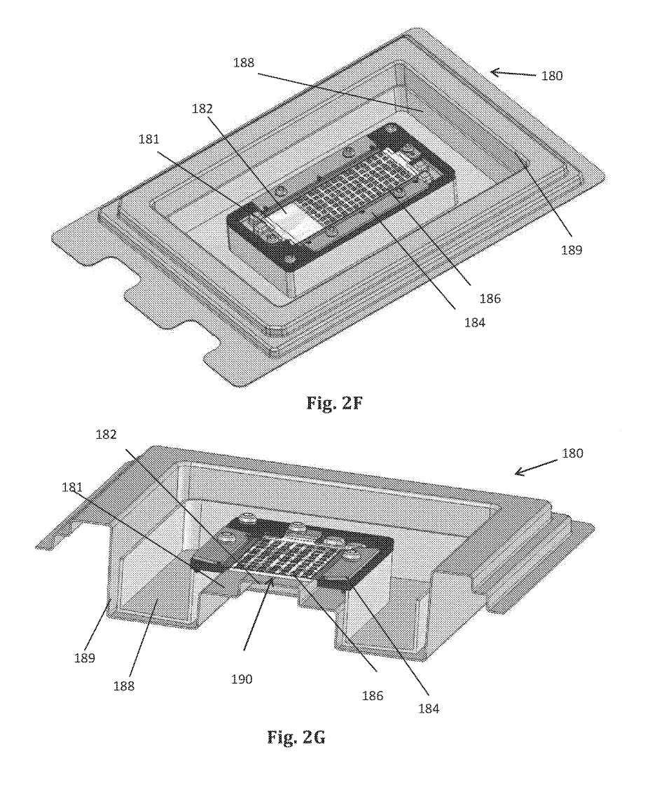

Reference is now made to FIG. 2A, which illustrates a perspective view of a casing configured to hold a substrate having a surface suitable for attachment of viable cells. As shown in FIG. 2A, substrate casing, 140 includes clamping elements (shown as elements 142A-C), configured to hold and secure the substrate (for example, a slide) to its location. Casing 140 forms a shallow region/space, 144, which allows drainage of excess liquid (such as cell medium) during the flooding process. In some embodiments, the walls of the casing, are higher than the substrate surface when it is positioned (in its groove), such that it can be immersed in medium while incubated.

Reference is now made to FIG. 2B, which illustrates a top perspective view of a mesh-holding frame configured to hold and stretch a mesh for further manipulation (such as depositing molecules on the mesh or positioning over a cell slide). Mesh frame, 150, in FIG. 2B is shown in the form of a rectangular frame, having an internal open space (152) over which the mesh may be placed/positioned/stretched. Mesh frame 150 may further include sealing stretching/fastening elements (shown as elements 154A-B), configured to provide uniform stretching of the mesh and to secure it in place.

In some embodiments, the substrate casing and the mesh frame may have similar or matching dimension, so as to allow alignment and fitting of the mesh frame (while carrying the mesh) and the substrate casing, such that when the two are approximated, the mesh, secured in the mesh frame may be aligned to the substrate held in the substrate casing, to result in alignment of molecule sets deposited on the mesh with cell groups attached to the substrate.

Reference is now made to FIG. 2C, which illustrates a top view of a substrate framed casing (holding a substrate) and a mesh-holding frame (holding a mesh), the mesh frame being positioned on top of the substrate casing. As shown in FIG. 2C, substrate casing 160, holds substrate 162 (shown in the form of a slide). Further shown is mesh-holding frame 164, positioned on substrate casing, such that mesh 166 (shown as gridded mesh) is aligned/positioned over the substrate, onto which cells are attached (not shown). The alignment/positioning of the substrate casing and the mesh-holding frame may be achieved by various means, such as, for example, but not limited to, visual means (for example, corresponding markers on each of the casing and frame), physical means (for example, matching grooves and protrusions, assuring alignment and correct positioning of the frame and casing), and the like. Upon positioning of the mesh carrying molecules over the substrate carrying cells, the molecules may be transferred to the cells, spontaneously or upon further manipulation, such as, for example, addition of a fluid. In some embodiments, the mesh and the cells may be incubated for any desired length of time, within the casing as long as the cells on the substrate are maintained in a hydrated state. In some embodiments, the mesh and the cells are incubated in the presence of a suitable fluid (such as, for example, but not limited to: cell medium, buffers, solutions, reagent mixes, and the likes or any combinations thereof). In some embodiments, the mesh-holding frame may further be used to promote separating the mesh from the substrate, with or without an attached float mechanism allowing such separation, without affecting the cells. In some embodiments, when a float is used, it may not necessarily be introduced following the incubation but attached to the mesh frame before it is placed on the slide.

In some embodiments, the framed substrate casing and/or the mesh-holding frame may be made of any suitable material. In some embodiments, they may be made of serializable material. According to some embodiments, the frame and/or casing are to withstand sterilizing procedures, such as, for example, an autoclave, chemiclav, gamma radiation, chemical sterilization, gas sterilization, a dry heat sterilizer, and the like. In some embodiments, the frame and/or casing may be made of low cost material(s).

Reference is now made to FIGS. 2D-E, which illustrate an exemplary float-device 201 configured for carrying/holding and separating a gridded mesh 214 from a substrate 212 without affecting the cells (for example, by pilling off cells, detaching the cells, breaking the cells, and the like). Float-device 201 having a mesh holder 204, and spacers 208.

FIG. 2D schematically illustrates yet another float-device in a cross section, according to some embodiments. Shown is float-device 201 hosting mesh 214 and substrate 212, respectively. The framed container holding the substrate and mesh is not shown. Capillary forces fasten mesh 214 to slide 212 when the float-device 201 is placed over the substrate, aligning the mesh 214 over substrate 212. According to some embodiments, spacers 208 are designed to prevent the float from dropping down all the way to the bottom of the substrate casing trench in the absence of liquid, thus creating too much pressure on the slide-mesh interface. Further shown are protruded rods, 204, designed to obstruct passage of the mesh. FIG. 2E schematically illustrates a cross section of float device 201 hosting substrate 212, separated from mesh 214, according to some embodiments. Throughout the separation, mesh 214 is carried by rods 204 as the float rises up. According to some embodiments, separation between mesh 214 and substrate 212 occurs by introducing a fluid configured to cancel out the capillary forces between mesh 214 and slide 212 thereby unfasten the connection between them as well as causing the float device 201 to rise up thus, lifting mesh 214 away from slide 212.

Reference is now made to FIGS. 2F-G which schematically illustrate the float device in the substrate casing, which facilitates separation between the mesh and the substrate, without harming the cells or molecules deposited on the substrate, according to some embodiments. FIG. 2F illustrates a perspective top view of a framed casing (180) which includes/holds a substrate (182) and a mesh-holding frame (184), holding a mesh (186), the mesh frame being positioned on top of the substrate and optionally being attached, connected to or associated with a flotation means (device) (188, shown as a rectangular floatation device). In some embodiments, the flotation means may be attached to the mesh frame permanently or transiently. In some embodiments, the flotation means may be an integral part of the mesh frame. In some embodiments, the flotation means may be placed in the substrate framed casing. In some embodiments, the float device while attached to the mesh frame may be placed at the bottom of the trench of the substrate framed casing during mesh-slide incubation in the absence of liquid. Reference is now made to FIG. 2G, which schematically illustrates a cross section view of the substrate casing (180), a mesh-holding frame (184), holding a mesh (186), the mesh frame optionally being attached, connected to or associated with a flotation means (device) (188, shown as a rectangular floatation device), during incubation with the slide and subsequently, as fluid is added to the interface (190) between the mesh and the substrate by dripping it on top the mesh. Addition of the fluid to the mesh-slide interface may cancel out capillary forces between the two and provide separation of the mesh from the substrate. In some embodiments, as fluid is added, the mesh may further separate and distant from the substrate. In some embodiments, the mesh may float away from the substrate. In some embodiments, excess liquid may be drained to trench (189), which results in separation of the mesh from the substrate, as facilitated by the flotation device (188) that lifts the mesh frame from the substrate, as excess fluid accumulates in the trench, lifting the float up. The separation of the mesh from the substrate is achieved without harming or otherwise affecting the cells on the substrate.

According to some embodiments, the fluid introduced for the separation process may be any water-based solution, such as an isotonic solution. For example, various cell culture media (such as DMEM eagle Earle's salts base, non-supplemented and/or supplemented with any additional ingredients) and/or buffers (such as PBS, TBS, and the like) may be used.

According to some embodiments, float device may be made of sterilizable material. According to some embodiments, float-device is configured to withstand sterilizing procedures, such as, for example, an autoclave, chemiclav, gamma radiation, chemical sterilization, gas sterilization, a dry heat sterilizer, and the like.

According to some embodiments, various interactions, such as, capillary forces allow the interaction/physical approximation between the molecules (for example, nucleic-acid molecules) on the mesh and the cells on the substrate. Advantageously, the use of the mesh for the indirect transfer of the sets of molecules to the cells provides a very efficient, accurate and cost effective manner to transfer the sets of molecules to the cells. In some exemplary embodiments, the use of said mesh with nucleic acid molecules for the indirect transfection of the cells results in a very efficient and accurate transfection of the cells on the substrate, due to the close physical proximity between the nucleic acid molecules (optionally in a composition comprising a transfection agent) and the target cells.

According to some embodiments, further provided are systems, kits, devices and methods for seeding viable cells on a substrate, using a seeding-mesh to deploy the viable cells onto the substrate. According to some embodiments, seeding viable cells to a surface of a suitable substrate using a seeding-mesh results in a cell-seeding pattern determined by the pattern/characteristics/structure of the mesh. In some embodiments, the cells are constrained by the threads of the mesh and availed through the holes (apertures) of the seeding-mesh to be deposited in a desired pattern on the substrate. Advantageously, the use of the seeding-mesh for the cell-seeding results in a homogeneous controllable seeding density across the surface of the substrate. In further embodiments, the use of such seeding mesh results in the cells being seeded in an array, which may advantageously be predetermined and/or addressable.

According to some embodiments, the present disclosure is further directed to systems, kits, devices and methods for transferring molecules to viable cells, in which the cells are seeded on a surface of a substrate in multiple groups (sets) with spatial separation there between, (i.e. in an array). The separation between the cell groups advantageously brings the ability to perform the transfer of different or multiple molecule sets to different or multiple cells groups while providing improved distinction between the different cell groups. Additionally, the seeding methods provided herein reduce cell stress which results in improved viability of the cells, improved transfection efficiency and uniformity and reduce side effects (such as cell density-derived biological background/noise) when using or testing the cells in downstream assays. In some embodiments, and without wishing to be bound to any theory or mechanism, the reduced stress may be achieved because the cells do not incur stress condition upon contact with the grid of the DNA mesh, as detailed below. According to some embodiments, seeding the cells on the surface according to the weaving pattern of the mesh advantageously provides homogeneity of cell densities across the surface within and between the different groups. According to some embodiments, the cell spot distinction and cell density homogeneity across the cell groups advantageously results in low deviations of results obtained when the cells are tested/used in downstream assays. In some embodiments, when nucleic acid molecules are used to transfect the cells in accordance with the systems, devices, kits and methods disclosed herein, the cell spot distinction and cell density homogeneity across the cell groups advantageously results in low deviations of transfection efficiency between different cell groups. Advantageously, the low deviation between the cell groups provides transfection results that are easily comparative either with reference groups or with other cell groups on the same substrate. Further, seeding cells in an array on the substrate enables the execution of various high-throughput assays which are very cost effective, accurate, reliable and reproducible.

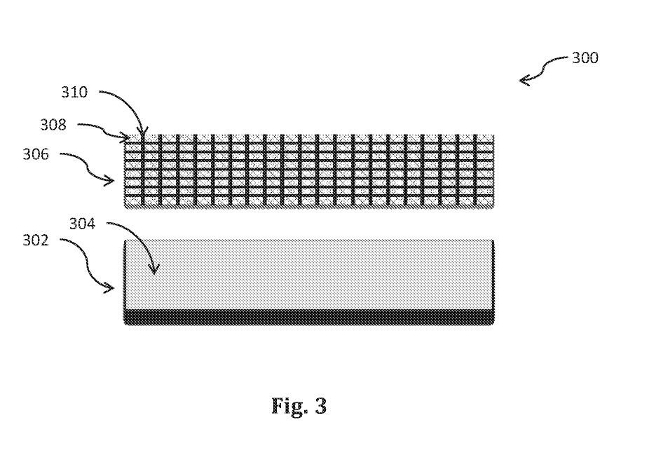

Reference is now made to FIG. 3, which schematically illustrates a cell-seeding kit 300, according to some embodiments. Cell-seeding kit includes a substrate 302 with a surface 304 suitable for adherence or attachment of viable cells, a seeding mesh 306 having seeding chambers 308 confined by a grid 310. Mesh 306 is configured to be placed on surface 304. Upon deposition of cell-containing solution, cell passage to surface 304 and therefore eventual seeding pattern/density is dictated by the weaving pattern of mesh 306. Grid 310 is configured to obstruct passage of viable cells in predetermined areas, thereby to confine seeding chambers 308. According to some embodiments, capillary forces are generated upon placing mesh 306 on surface 304 in the presence of fluid, resulting in fastening mesh 306 to surface 304.

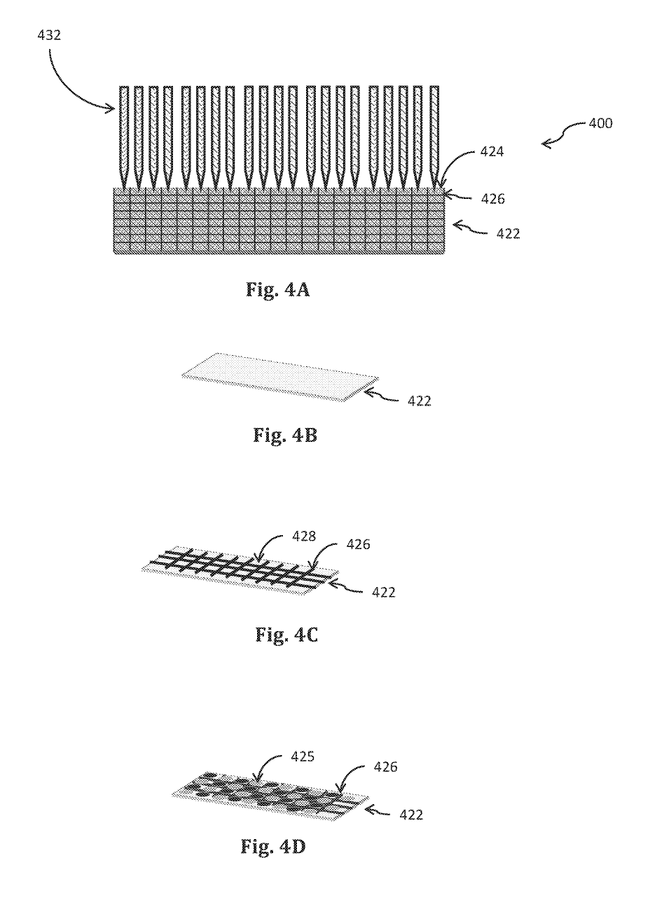

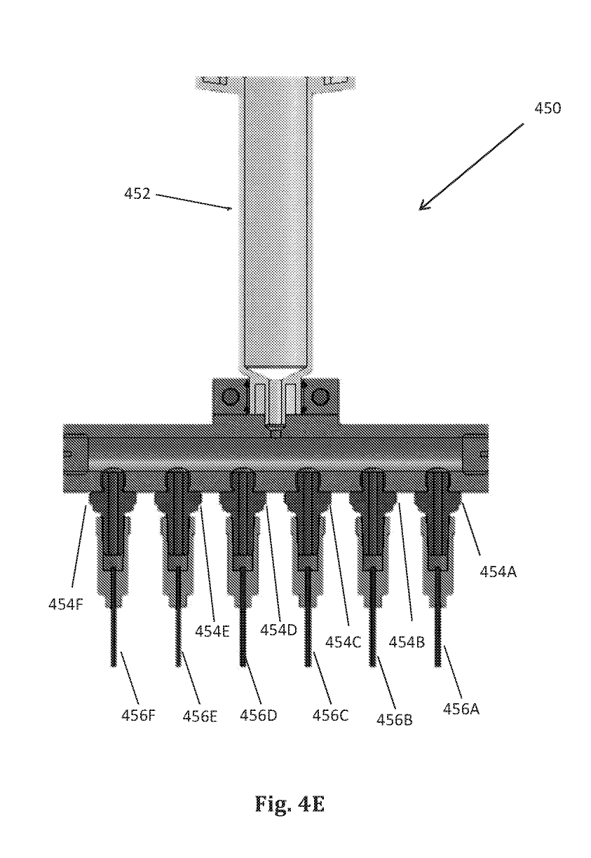

Reference is now made to FIGS. 4A-D which illustrates molecules transferring (printing) kit 400, according to some embodiments. Printing kit 400 includes a mesh 422, a constraining grid 426, an array of grid-free chambers 424 and a suitable molecule printer/dispenser 432, which may have one or more printing tips. In some embodiments, the dispenser may be operated manually, semi-automatically or automatically.

FIG. 4A illustrates a molecule dispenser (printer) 432 deploying molecules to multiple grid-free chambers 424 confined by grid 426 on mesh 422. According to some embodiments, molecule printer 432 is configured to controllably deploy a plurality of predetermined molecule types, or other solutions such as cell suspension solution or cell medium. According to some embodiments, molecule printer 432 may be configured to deploy a fluid containing molecules to each printing chamber 424 in varying volumes.

According to some exemplary embodiments, when the printed molecules are nucleic-acid molecules, they may be contained in a transfection mix or buffer. In some exemplary embodiments, the transfection mix may include such components as, but not limited to: transfection reagent (such as, for example, Lipofectamine, Transfectamine, Effectene, Fugene, PolyJet, JetPEI, PEI, and the like), buffer (such as, for example, NaCl solution, Effectene kit EC buffer, OptiMem, and the like), additional agents (such as, for example, Effectene kit enhancer, sucrose, gelatin, and the like), or combinations thereof. Each possibility is a separate embodiment.

FIG. 4B illustrates mesh 422 prior to gridding thereof. FIG. 4C illustrates mesh 422 including grid 426 confining printing chambers 428.

FIG. 4D illustrates mesh 422 including grid 426 confining printing chambers with multiple molecule sets 425 printed on mesh 422, each set is printed on a different printing spot/chambers.

Reference is now made to FIG. 4E, which illustrates a perspective front view of an exemplary solution dispenser (printer), according to some embodiments. Exemplary dispenser 450 includes a cartridge 452, configured to allow maneuvering/operation of the dispenser and to optionally further hold molecules or molecules solutions (for example a solution of nucleic-acids) to be dispensed on the substrate. Dispenser 450 further includes one or more separable printing tips/nozzles (shown as printing tips 454A-F). In some embodiments, the tips may be permanent or disposable. In some embodiments, the tips may have disposable, replaceable ends, configured to be reversibly situated on the end of the tip. Shown in FIG. 4E exemplary disposable tip ends 456A-F, situated on the respective tips, 454A-F. In some embodiments, the printing tips may be identical or different from one another in structure, composition and operation. In some embodiments, the tips may operate simultaneously in a different, similar or identical manner. Each tip may dispense the same type of molecule or different types of molecules, depending on the setting of the dispenser and if/what type of reservoir is used. In some embodiments, each tip may dispense an equal amount/volume of molecules. In some embodiments, each tip may dispense a different amount/volume of molecules. In some embodiments, the dispenser tip(s) are positioned so as to align with matching printing chambers (situated in an array), such that the type and/or composition and/or the amount/concentration of the molecules dispensed to each chamber is known and addressable.

According to some embodiments, there is provided an indirect transfection system that may include a slide having a cell carrying surface configured to carry viable cells; a plurality of viable cell groups located at predetermined distinct locations on the cell carrying surface, wherein the cell carrying surface comprises cell-free space configured to provide spatial separation between the cell groups; a mesh configured to carry nucleic-acid molecules; and a plurality of nucleic-acid molecule sets located at predetermined distinct locations on the mesh wherein the mesh comprises nucleic-acid molecule-free space configured to provide spatial separation between the nucleic-acid molecule sets; wherein the locations of the viable cell groups match the locations of the nucleic-acid molecule sets such that upon approximating the cell carrying surface and the mesh, at least some nucleic-acid molecule sets are introduced to at least some matching viable cell groups.

According to some embodiments, there is provided an indirect transfection system that may include a substrate having a cell carrying surface configured to carry viable cells; the substrate may have a planar, flat or semi-flat surface configured to carry, hold, attach viable cells; a plurality of viable cell groups disjointedly seeded at known distinct locations on the surface, wherein on the surface there are cell-free areas providing spatial separation between different cell groups; a mesh configured to carry nucleic-acid molecules in a controllably releasable manner; and a plurality of nucleic-acid molecule sets absorbed within the mesh in distinct locations. The mesh has nucleic-acid molecule-free space providing spatial separation between different nucleic-acid molecule sets; wherein the location of the nucleic-acid sets and the cell groups are such that, upon placing the mesh on the surface, at least some nucleic-acid molecule sets are introduced to at least some cell groups.

In some embodiments, the mesh is configured to develop capillary forces with the surface of the slide.

In some embodiments, the nucleic-acid molecule-free space comprises a hydrophobic material soaked within the mesh. The hydrophobic material is configured to repel/repulse water-based solutions (including nucleic-acid molecule containing solutions), to thereby provide nucleic-acid molecule-free space within the mesh and nucleic-acid absorbing chambers confined by the hydrophobic material.

According to some embodiments, the nucleic-acid molecule-free space comprises a hydrophobic polymer absorbed within the mesh, the hydrophobic polymer is configured to repel/repulse water-based solutions (including nucleic-acid molecule containing solutions), thereby provide nucleic-acid molecule-free space within the mesh and nucleic-acid absorbing chambers confined by the hydrophobic polymer.

According to some embodiments, the hydrophobic polymer is arranged in horizontal and vertical lines forming a hydrophobic polymer matrix such that the nucleic-acid absorbing chambers are rectangular spots/chambers confined by the horizontal and vertical lines, or any desired shape.

According to some embodiments, the hydrophobic polymer forms a grid such that the nucleic-acid absorbing chambers are confined in a matrix/array. In some embodiments the array may be an addressable array, a predetermined array, and/or a designated array.

According to some embodiments, the cell-free space comprises cell-free horizontal and vertical lines forming a cell-free grid such that cell groups are arranged in rectangular shapes confined/bordered by the cell-free horizontal and vertical lines.

According to some embodiments, the cell groups are positioned/located on the surface in a matrix pattern with a cell-free space grid providing separation between the groups, to form an array. In some embodiments, the array may be an addressable array, a predetermined array, and/or a designated array.

According to some embodiments, at least some of the nucleic-acid retaining chambers and the cell groups are co-centric such that when the mesh and the slide are approximated at least some centers of some nucleic-acid retaining chambers and some centers of some cell groups match.

According to some embodiments, the matrix/array of the nucleic-acid retaining chambers is configured to match the matrix/array of the cell groups such that upon placing the mesh on the surface of the slide, at least some nucleic-acid molecule sets and at least some cell groups are introduced optionally in a co-centric manner.

According to some embodiments, upon placing the mesh on the surface of the slide, the cell-free grid is configured to overlap the nucleic-acid molecule grid such that, for at least some cell groups, placing the mesh on the surface does not expose them to the nucleic-acid molecule grid.

In some embodiments, the system may include a frame for maintaining the orientation of the molecules mesh in alignment with the cells seeding surface. In some embodiments the system may include a frame for positioning/placing/stretching the molecules mesh such that the molecules deposited on it encounter an essentially flat uniform interface. In some embodiments, the system may include a framed container configured to hold the cell seeding substrate. The mesh holding frame aligns over the substrate while in the framed casing and facilitates detaching of the mesh from the seeding surface, after the molecules have transferred to the cells on the substrate, essentially without affecting the cells. In some embodiments, the frames may be separate frames or one frame configured to enable one or more of the above mentioned configurations.

In some embodiments, the system may further include a mesh-holding frame configured to facilitate detaching of said molecules mesh from the cells on the essentially without harming the cells, without pilling the cells, without affecting the dispersion of the cells on the substrate, or without otherwise affecting the cells in any manner.

According to some embodiments, there is provided an indirect transfection system, comprising: a cell-slide having a surface configured to carry viable cells, a plurality viable cells deposited on said surface, a mesh configured to carry nucleic-acid molecules and multiple nucleic-acid molecule sets located at predetermined locations on said mesh.

According to some embodiments, the mesh is configured to be placed on the surface of the substrate and release/deploy at least some of said nucleic-acid molecule sets on said surface to be transfected to at least some of said viable cells.

According to some embodiments, the mesh is configured to be fastened on said surface by capillary forces.

According to some embodiments, the viable cells are homogeneously deposited on said surface.

According to some embodiments, the viable cells are deposited on said surface in multiple disjoint cell groups at different locations on said slide with homogeneity in cell distribution between said groups.

In some embodiments, the cells are situated on the substrate in controlled/homogenous cell seeding density, within the predetermined array (i.e. within each chamber of the array).

According to some embodiments, the mesh may be extruded, oriented, expanded, woven or tubular; the mesh may be made from connected strands of polymers or other inert materials that define a mesh structure with a mesh pattern confining the plurality of holes/apertures in the mesh. According to some embodiments, the mesh may have a weaving pattern confining the holes thereof. According to some embodiments, the mesh may have a lattice structure confining the holes thereof. According to some embodiments, a mesh may be a web, a net, a lattice, a honey-comb, a matrix, and the like.

According to some embodiments, there is provided a nucleic-acid printing device, that may include a mesh configured to carry nucleic-acid molecules; a restrainer at least partially absorbed within the mesh at predetermined locations, the restrainer is configured to repel/repulse water-based solutions (such as nucleic-acid containing solutions), thereby provide nucleic-acid molecule-free spaces within the mesh and nucleic-acid retaining chambers confined by the restrainer, in the form of an array; and a nucleic-acid molecule printer configured to deploy nucleic-acid molecules to the nucleic-acid retaining chambers. In some embodiments, the device may further include a mesh-holding frame, configured to hold/stretch the mesh such that the molecules may be printed thereupon. In some embodiments, the mesh-holding frame may further be used for further manipulation of the mesh, such as positioning the mesh in alignment over a substrate.

According to some embodiments, there is provided a transfection system, that may include a substrate which may have a surface configured to carry/promote attachment of viable cells; a gridded mesh, attached on the surface of the substrate by capillary forces and configured to either avail deployment of viable cells to the slide or avail indirect transfection of multiple nucleic-acid molecule sets; and a frame, configured to hold the gridded mesh, align it over the slide and to optionally provide a float structure designed to promote cell pilling-free separation between the gridded mesh and the substrate upon the introduction of fluids. In some embodiments, the mesh is removed from the system, without affecting the cells.

According to some embodiments, there is provided a method for transferring molecules, such as, nucleic acid molecules, proteins, peptides, antibodies, enzymes, lipids, small organic molecules, beads, polymers, metals and the like, onto and/or into viable cells, the method may include one or more of the following steps:
a. Providing a mesh comprising a plurality of molecules or molecule sets deposited/absorbed/retained or/within the mesh at predetermined disjoint locations separated by a molecule free space (i.e. in an array);
b. Providing a substrate having a seeding surface, capable of promoting attachment of/attaching to viable cells, and a plurality of viable cells or cell groups disjointedly attached on the seeding surface at predetermined locations, preferably matching with the predetermined locations of the molecule sets on the mesh and the cell-free space separating the cell groups;
c. Approximating the mesh to the cells on the seeding surface such that at least some molecule sets are transferred to matching cell sets;
d. Incubating the cells on the substrate with the mesh; and
e. Separating the mesh from the cells on the substrate, thereby obtaining a substrate with multiple sets of cells having molecules transferred thereto.

According to some embodiments, there is provided a method for transferring molecules into cells, the method may include one or more of the following steps:
a) providing a mesh comprising molecules deposited on said mesh in a designated array;
b) providing a substrate comprising a surface deposited with viable cells in an array of predetermined locations;
c) approximating a surface of the mesh to the viable cells on the substrate surface such that at least some molecules are introduced to at least some of the viable cells, thereby transferring the molecules to at least some of the viable cells; In some embodiments, the transfer may be facilitated by capillary forces generated at the slide-mesh interface;
d) incubating the mesh with the viable cells on the substrate;
e) separating the mesh from the cells on the substrate, thereby obtaining a substrate with multiple sets of cells having molecules transferred thereto.

In some embodiments, separating/removing/detaching the mesh from the multiple sets of cells on the substrate does not affect the cells (for example, does not harm the cells, does not pill-off the cells, does not disturb the cells pattern on the substrate, and the like).

According to some embodiments, there is provided a method for transfecting nucleic-acid molecules into viable cells, the method may include one or more of the following steps:
a. Providing a mesh comprising a plurality of nucleic-acid molecule sets absorbed within said mesh at predetermined disjoint locations separated by a nucleic-acid free space (i.e. in an array);
b. Providing a substrate (such as a slide) with a seeding surface, configured to allow or promote attachment of viable cells, and a plurality of viable cell groups disjointedly attached on the seeding surface at predetermined locations matching with the predetermined locations of the nucleic-acid molecule sets on the mesh and cell-free space separating the cell groups;
c. Approximating the mesh to the cells on the seeding surface such that at least some nucleic-acid molecule sets are introduced to matching cell sets;
d. Incubating the cells on the substrate with the mesh; and
e. Separating the mesh from the slide, thereby obtaining a substrate with a plurality of transfected cell groups.

In some embodiments, separating the mesh from the substrate does not affect the plurality of cell groups on the substrate.

According to some embodiments, there is provided a method for transfecting nucleic-acid molecules into cells, the method may include one or more of the following steps:
a) providing a mesh comprising nucleic-acid molecules deposited on said mesh in a designated array;
b) providing a substrate comprising a surface deposited with viable cells in an array of predetermined locations;
c) approximating a surface of the mesh to the viable cells on the substrate surface such that at least some nucleic-acid molecules are introduced to at least some of the viable cells, thereby transfecting at least some of the viable cells;
d) incubating the viable cells on the substrate with the mesh; and
e) separating/removing/detaching the mesh from the cells on the substrate, thereby obtaining a substrate with multiple sets of transfected cells

According to some embodiments, the nucleic-acid mesh further comprises a nucleic-acid molecule restrainer located within the mesh and configured to separate between the nucleic-acid molecule sets. In some embodiments, the restrainer comprises a polymer arranged in a grid structure. In some embodiments the polymer is hydrophobic polymer. In some embodiments, the grid may be made of a liquid or semi-liquid hydrophobic material, capable of solidifying. In some embodiments, polymer may be a thermoplastic polymer, thermally-cured, liquid soluble polymer, a photo-initiated polymer, non-toxic hydrophobic material, and the like. In some embodiments, the grid is firm after solidifying. In some embodiments, the grid material is non-toxic to the cells. In some embodiments, removing the mesh from the cells does not affect the cells.

According to some embodiments, the viable cell groups are located on the seeding surface such that, upon approximating the nucleic-acid mesh to the seeding surface, the restrainer is introduced to an area within the cell-free space.

According to some embodiments, there is provided a transfecting method for nucleic-acid molecules into viable cells, the method includes the steps of: providing a mesh comprising a plurality of nucleic-acid molecule sets and/or transfection mixes absorbed and/or retained within said mesh, providing a substrate with a seeding surface, configured to carry viable cells, and multiple viable cells attached to the seeding surface, and approximating the substrate to the seeding surface such that at least some nucleic-acid molecule sets are introduced to viable cells.

According to some embodiments, the systems, devices, kits and methods provided herein can be used for transferring various solutions from a mesh retaining such solutions to a suitable substrate. In some embodiments, the solutions may containing various types of molecules, such as, but not limited to: proteins, peptides, antibodies, antigens, enzymes, lipids, beads, polymers, metals, organic molecules and the like. Each possibility is a separate embodiment. Exemplary application include the transferring of labeled antibodies onto viable cells or cell extracts, seeded on a corresponding substrate. Additional exemplary application include the transferring of various enzymes onto viable cells or cell extracts, seeded on a corresponding substrate. Additional exemplary application include the transferring of receptor ligands or enzyme substrate onto viable cells or cell extracts, seeded on a corresponding substrate.

In some embodiments, the systems, devices, kits and methods provided herein can be used for transferring of one type of molecule onto a second type of molecule. In some embodiments, this may allow identifying or screening for various interactions between the two types of molecules. In some exemplary embodiments, the systems, devices, kits and methods provided herein can be used for the formation of an array of antibodies deposited on a suitable slide, and array of ligands/biological sample/cell extracts transferred thereto via a mesh (or vice versa). In some exemplary embodiments, the systems, devices, kits and methods provided herein can be used for deployment of multiple different cell lysates (extracts) via a mesh onto a substrate surface which carries reactive compounds (for example, a suitable enzyme) to identify which cell lysate includes an ingredient capable of reacting with the enzyme (or vice versa, depositing cell extract on the slide and exposing it to a variety of enzymes). In some exemplary embodiments, the systems, devices, kits and methods provided herein can be used for generating a 2-way-hybrid system, to identify interaction between a first substance and a second substance. In such embodiments, an array of a first substance is formed on a substrate and interacted with a mesh deposited with an array of a second substance. Thereafter, potential interaction may be identified, for example, by fluorescence as a readout for binding or reactivity between the first substance and the second substance.

According to some embodiments, there is provided a system for depositing a second substance on a first substance, the system comprising: a substrate having a surface containing, deposited with, coated with or formed with a first substance; a mesh configured to carry multiple sets of a second substance arranged in a designated array, such that said sets are separated from each other, wherein said mesh is configured to be placed on said surface and deposit at least some of said second substance on at least some of said first substance, while maintaining spatial separation of or between the sets within the designated array; and a dispenser configured to dispense said second substance sets onto said mesh according to the designated array. In some embodiments, the first substance and/or the second substance may each be independently selected from cells, peptides, enzymes, antibodies, ligands, proteins, nucleic acid molecules, small organic molecule, organic molecules, lipids, beads, metals, polymers, and the like. Each possibility is a separate embodiment.

In some embodiments, the first substance may homogeneously coat, deposited on or formed with the surface. In some embodiments, the designated array may be defined by a grid on said mesh.

In some embodiments, the first substance may be divided to a plurality of groups, wherein said groups are separated from each other, wherein locations of said groups correspond to locations of said sets, such that upon approximating said surface and said mesh, at least some sets are introduced to at least some groups, such that a single set is introduced to a single group. In some embodiments, the mesh is removed from the substrate, without affecting the first and/or second substances (for example, without affecting the dispersion or the spatial separation on the substrate).

According to some embodiments, there is provided a kit for depositing or transferring a second substance on a first substance, the kit may include: a substrate having a surface containing, deposited with, coated with or formed with a first substance; and a mesh carrying multiple sets of a second substance arranged in a designated array, such that said sets are separated from each other, wherein said mesh is configured to be placed on said surface and deposit at least some of said second substance on at least some of said first substance, thereby allow interaction between said first and said second substances, while maintaining spatial separation of the sets within the designated array.

According to some embodiments, there is provided a kit for depositing or transferring a second substance on a first substance, the kit comprising: a mesh carrying multiple sets of a second substance arranged in a designated array, such that said sets are separated from each other, wherein said mesh is configured to be placed on a surface containing, deposited with, coated with or formed with a first substance and deposit at least some of said second substance on at least some of said first substance, thereby allowing interaction between said first and said second substances, while maintaining spatial separation of the sets within the designated array. In some embodiments, the mesh is removed without affecting the sets on the substrate.

According to some embodiments, there is provided a method for depositing or transferring a second substance onto a first substance, the method comprising:
a) providing a substrate having a surface containing, deposited with, coated with or formed with a first substance;
b) providing a mesh carrying multiple sets of a second substance arranged in a designated array, such that said sets are separated from each other; and
c) approximating the mesh to be aligned with the surface such that at least some of the second substance is released from the mesh and introduced to at least some of the second substance.

In some embodiments, the first substance may be homogeneously distributed over the surface.

In some embodiments, the first substance may be divided to a plurality of groups, wherein said groups are separated from each other, wherein locations of said groups correspond to locations of said sets, such that upon approximating said surface and said mesh, at least some sets are introduced to at least some matching groups, such that a single set is introduced to a single group.

In some embodiments, the method may further include dispensing the multiple sets of the second substance in a designated array on the mesh.

In some embodiments, the method may further include separating the mesh from the substrate, thereby obtaining a substrate comprising the second substance deposited on the first substance. In some embodiments, separating the mesh does not affect the first and/or second substances.

In some embodiments, the substrate surface may be coated prior to being deposited with the first substance. According to some embodiments, the coating layer is homogeneously coated, deposited on or formed with said surface. In some embodiments, the coating may be selected from, but not limited to: hydrogel, epoxysilane, aldehydesilane, streptavidin, silane, epoxide, maleimide, and the like, or combinations thereof. Each possibility is a separate embodiment.

According to some embodiments, the systems and devices disclosed herein may utilize one or more automatic or semi-automatic means/applicators. For example, depositing/dispensing/printing of cells, sets of molecules and/or grids may be performed by such automated or semi-automated dispensers, printers and/or applicators, each capable of applying a desired amount/concentration/volume of a desired cell, molecule or substance at a desired location in a an accurate manner.

According to some embodiments, one or more of the steps in the methods disclosed herein may be performed by a suitable automated or semi-automated system. For example, depositing/dispensing/printing of cells, sets of molecules and/or grid lines may be performed by one or more such systems.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope

### EXAMPLES

### Example 1: Cell Seeding

Exemplified herein is a cell seeding method for achieving inter-slide and intra-slide homogeneity of cell seeding by using a polymeric mesh at the cell-slide (substrate) interface.

According to the example, seeding is performed using a 100 μm nylon mesh (Merk Millipore, cat no. NY1H00010) stretched over a dedicated mesh-frame. The mesh is printed with vertical and horizontal liquid hydrophobic, thermoplastic material (PVC-based) lines, which are non-toxic to cells, to form an array of chambers (seeding spaces/spots) each confined by the vertical and horizontal hydrophobic lines. For example, the thickness (width) of the printed lines on the mesh may be 1.5 mm generating 2 mm×2 mm square chambers (seeding spaces/spots) of about 3.5 mm pitch. The mesh may be then baked for 20 min in an oven pre-heated to 100° C.

A Poly-L-Lysine coated slide (Polysciences cat no. 22247) is positioned in a substrate carrying case.

Full medium may be dripped on the upper side of the seeding-mesh at this stage, or at a later stage, as detailed below. The medium may be any suitable medium, depending on the type of cell and downstream assay. In one example, the medium is MEM eagle Earle's salts base supplemented with 10% FBS, 1× Pen-strep solution, 1 mM Sodium Pyrovate and 2 mM L-glutamine; (Biological Industries, cat no. 01-040-1A, cat no. 04-127-1A cat no. 03-031-1B cat no. 03-042-1B, cat no. 03-020-1B, respectively).

Then, mesh-holding frame carrying the mesh is placed/aligned within the substrate carrying case, such that it is exactly aligned with the designated contours of the substrate carrying case and hence aligned with the substrate. After the substrate and mesh are aligned; 100-400 μl of full medium is dispensed over the upper side of the mesh.

Next, cell suspension is dispensed. In one example, Hela cells are seeded at about 1*10⁴ cells/μl (=10⁶ cells/ml) suspension in full medium by automated means; ˜350 nl/chamber. The cell suspension is dispensed to the center of each chamber in the array. The cell suspension may be dispensed by manual or semi-automatic means, in which case, the volume and/or amount of cells dispensed to the chamber may be higher (for example, 1 μl/chamber).

After the seeding step, the substrate is incubated for 30 minutes at 37° C.

Then, 2-3 ml of full medium is dripped on the top of the mesh such that the slide-mesh interface is flooded, allowing the mesh to float above the slide such that it may be removed without pilling off cells. Then, incubation at 37° C. is carried until use in downstream assays.

### Experimental Protocol of Example 1

1. Stretch a 100 μm nylon mesh (Merk Millipore, cat no. NY1H00010) in a mesh-holding frame.

2. Grid polymer squares (1.5 mm line width using 18 mm/sec dispenser motion speed; spacing of 2 mm×2 mm using a pitch/offset of 3.5 mm; 6×13 array chambers according to the dimensions of the clear area on the substrate) on the 100 μm nylon mesh (Merk Millipore, cat no. NY1H00010) with liquid, non-toxic hydrophobic material.

2. Bake the meshes for 20 mins in an oven pre-heated to 100° C.

3. Place a Poly-L-Lysine coated slide (Polysciences cat no. 22247) in the substrate (slide) casing.

4. Place the mesh-holding frame such that it is exactly aligned with the contour of the coated slide.

5. Gently drip on top the mesh 100 μl of full medium (MEM eagle Earle's salts base supplemented with 10% FBS, 1× Pen-strep solution, 1 mM Sodium Pyrovate and 2 mM L-glutamine; Biological Industries, cat no. 01-040-1A, cat no. 04-127-1A cat no. 03-031-1B cat no. 03-042-1B, cat no. 03-020-1B, respectively). Wait for the liquid to spread through the mesh.

6. Above the center of each mesh chamber, seed Hela cells at 1*10⁴ cells/μl suspension in full medium; 0.35 μl drop per hole (automatically dispensing at: 0.05 sec/spot, 1 Bar air pressure, 1 cc syringe, 0.16 mm inner diameter needle).

7. Incubate cell slides with the mesh in an incubator at 37° C. for 30 minutes.

8. Flood mesh-slide interface with full medium such that capillary forces between them are eliminated. Gently remove the floating mesh.

9. Incubate at 37° C. until use. Preferably, an overnight incubation.

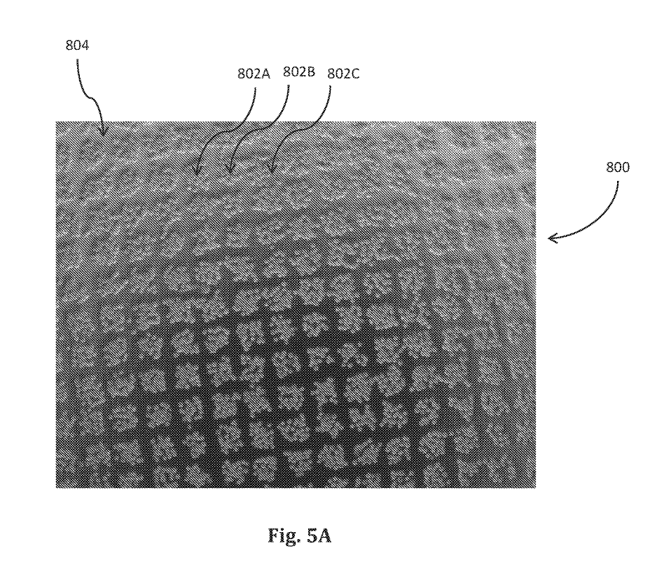

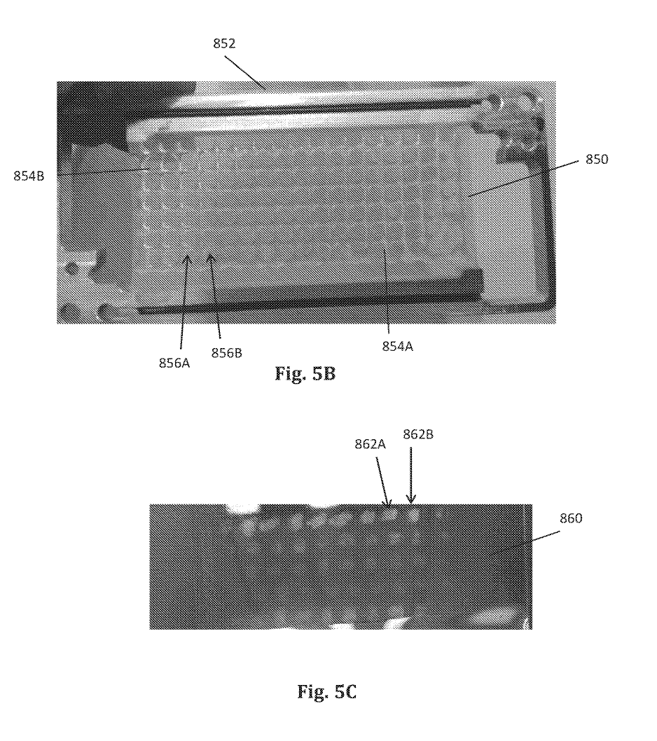

The results of the seeding performed as described herein are presented in FIG. 5A, which shows a pictogram of part of a cell slide (800), which carries multiple cells (shown as exemplary cells 802A-C), seeded at high degree of order and uniform density according to the pattern of the seeding mesh (weaving pattern of a 100 μm nylon mesh in this example). This represents the type of homogeneous seeding density present within each cell spot of the array. FIG. 5B shows a pictogram of a mesh (850), placed on a mesh holder (852) and printed with grid lines (such as exemplary representative gridlines 854A-B are indicated) having a thickness/width of 1.5 mm to form chambers (such as exemplary representative chambers 856A-B which are indicated) having dimensions of 2 mm². FIG. 5C shows a pictogram of 2 mm² spots of cell groups (shown as representative cell groups 862A-B) seeded on a substrate (a transparent coated glass slide (860)), using the mesh of FIG. 5B.

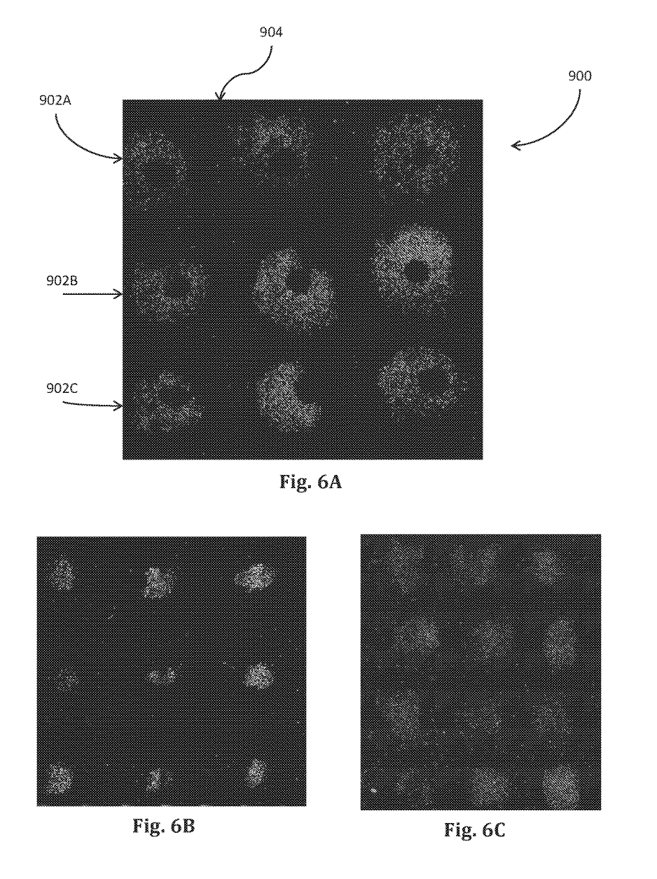

The results presented in FIG. 6A show pictogram of part of a surface of a substrate (shown as slide 900) showing groups of cells (shown as exemplary groups 902A-C), generated by seeding cell suspension solution through a 100 μm nylon mesh gridded by the hydrophobic polymer. The polymer grid dictated the cluster pattern wherein the cells are spatially separated by cell-free area (for example, 904). Similarly, the results presented in FIGS. 6B-C show pictograms of part of a surface of a substrate seeded with lower amount of cells (FIG. 6B) or higher amount of cells (FIG. 6C). The cells shown in FIG. 6A were seeded manually (i.e., cell suspension was dripped manually), and the cells shown in FIGS. 6B-C were seeded by an automatic cell dispenser.

### Example 2: Nucleic Acid Printing Method and Indirect Transfection of Seeded Cells

Exemplified in this example is a nucleic-acid printing method for generating a mesh carrying separable sets of nucleic acid molecules in an array pattern, and its use for parallel transfection of multiple cell groups.

According to the example, a nucleic-acid mesh is stretched over a dedicated frame. The mesh in this examples is a 41 μm nylon mesh (for example Merck Millipore, cat no. NY4100010). The mesh is patterned by vertical and horizontal 1 mm thick lines of liquid non-toxic hydrophobic material generating 2.5 mm×2.5 mm chambers (printing spaces/spots) confined by the vertical and horizontal hydrophobic lines. The mesh is then baked for a duration of 20 min in an oven pre-heated to 100° C. to solidify the polymer pattern.

Specific transfection mix(s) are prepared using Effectene transfection kit (Qiagene, cat no. 301425), according to the following protocol: Briefly, 3.25 μl buffer EC+0.6 μl sucrose 1.5 mM+0.5 μl DNA are mixed. 4 μl Enhancer and 5 μl Effectene are added and incubated at room temperature for 15 minutes. 6 μl EC buffer are added.

The mixtures are then automatically dispensed/dripped onto each chamber of the gridded mesh, after verifying alignment of the dispenser and the array on the mesh. Dispensing may be performed manually or using an automated or semi-automated dispenser.

For dry transfection, the DNA printed mesh is placed in a desiccator and stored at 4° C. until use. For wet transfection, the printing may be performed in a humid environment (in a climate controlled chamber) in order to prevent the transfection mix(s) from drying. After printing, the mesh-holding frame is transferred to humidity chambers until used for transfection.

Prior to approximating the nucleic-acid mesh and the cells-carrying substrate, the cell substrate is prepared for transfection by eliminating most fluids from the carrying case except for fluids retained by the cells themselves, for example, by using vacuum-based aspiration. Then, the mesh-holding frame is aligned and place over the cells on the substrate, by aligning the frame and the substrate casing, such that each printed chamber is located above a seeded spot; printed side of the mesh faced down. Then, 0.3 μl of antibiotics-free medium (MEM eagle Earle's salts base supplemented with 10% FBS, 1 mM Sodium Pyrovate and 2 mM L-glutamine; Biological Industries, cat no. 01-040-1A, cat no. 04-127-1A, cat no. 03-042-1B, cat no. 03-020-1B, respectively) is added using an automatic dispenser above each chamber, followed by 0.3-3 hours of incubation at 37° C.

To separate the mesh from the slide, the slide-mesh interface is flooded with medium to cancel out capillary forces fastening the mesh to the slide. Then the mesh frame (with the mesh) is gently lifted from the substrate.

Antibiotics-free medium is added to immerse the top surface of the slide.

The slide is then incubated for 12-48 hours at 37° C.

The cells may then be optionally fixed to the surface of the slide for preservation and for further processing and analysis in downstream assays.

### Experimental Protocol of Example 2

1. Stretch a 41 μm nylon mesh (Merk Millipore, cat no. NY4100010) in a mesh-holding frame.

2. Grid polymer squares (1 mm line width using 40 mm/sec dispenser motion speed; spacing of 2.5 mm×2.5 mm using a pitch/offset of 3.5 mm; 6×13 array of chambers according to the dimensions of the clear area on the cell substrate) on 41 μm nylon mesh (Merk Millipore, cat no. NY4100010) with liquid hydrophobic non-toxic material, while the mesh is placed in a frame. Bake the meshes for 20 mins in an oven pre-heated to 100° C.

3. Prepare DNA-specific transfection mix(s) using Effectene transfection kit (Qiagene, cat no. 301425): combine 3.25 μl buffer EC+0.6 μl sucrose 1.5 mM+0.5 μl DNA (total quantity for both reporter gene and tested gene DNA), add 4 μl Enhancer, add 5 μl Effectene and incubate at Room-Temperature for 15 minutes. Add 6 μl EC buffer.

4. Drip 0.3 μl of the above mix onto each chamber of the gridded 41 μm mesh (automatically dispensing at: 0.05 sec/spot, 1 Bar air pressure, 1 cc syringe, 0.16 mm inner diameter needle). For wet transfection, the printing is performed in a humid environment in order to prevent the meshes from drying.

5. Immediately after printing, prepare cell seeded substrate for transfection by tilting the substrate casing and aspirating medium avoiding the top face of the slide. Slide-mesh contact is performed in a humid environment.

6. Gently align and place the mesh-holding frame with the nucleic acid-printed mesh on the slide surface while in the substrate casing, over the cells—each printed chamber above a seeded spot;

7. Drip over each mesh chamber 0.3 μl of no P/S medium (MEM eagle Earle's salts base supplemented with 10% FBS, 1 mM Sodium Pyrovate and 2 mM L-glutamine; Biological Industries, cat no. 01-040-1A, cat no. 04-127-1A, cat no. 03-042-1B, cat no. 03-020-1B, respectively) (automatically dispensing at: 0.05 sec/spot, 1 Bar air pressure, 1 cc syringe, 0.16 mm inner diameter needle), and incubate for 20 mins at 37° C.

8. Flood slide-mesh interface by applying 2-3 ml of full medium on top the mesh while still on top the slide. Remove mesh.

9. Incubate at 37° C. for an additional 12-72 hrs using the following protocol: 6 hrs post transfection initiation medium is replaced to empty medium (MEM eagle Earle's salts base supplemented with 1× Pen-strep solution; Biological Industries, cat no. 01-040-1A, cat no. 03-031-1B, respectively).

10. Fix the cells on the slide using the following protocol: Aspirate medium from the carrying case; wash with 5 ml of PBS (Biological Industries, cat no. 02-020-1A), aspirate PBS; Gently drip 0.5 ml of fresh 4% PFA solution (1:4 16% PFA solution in PBS supplemented with 6.3% D-glucose; EMS cat no 15710, Biological Industries, cat no. 02-020-1A) on the seeded area of the slide; Incubate for 10 mins; Aspirate, wash twice with PBS; Gently drip 0.5 ml of fresh DAPI solution (1:10000 1 mg/ml DAPI solution in DDW) on the seeded area of the slide; Incubate for 5 mins; aspirate and wash with 10 ml of PBS.

In order to test the transfection efficiency and accuracy, the cells are imaged for expression of an exogenous fluorescent protein, the nucleic acid encoding thereto transfected to the cells.

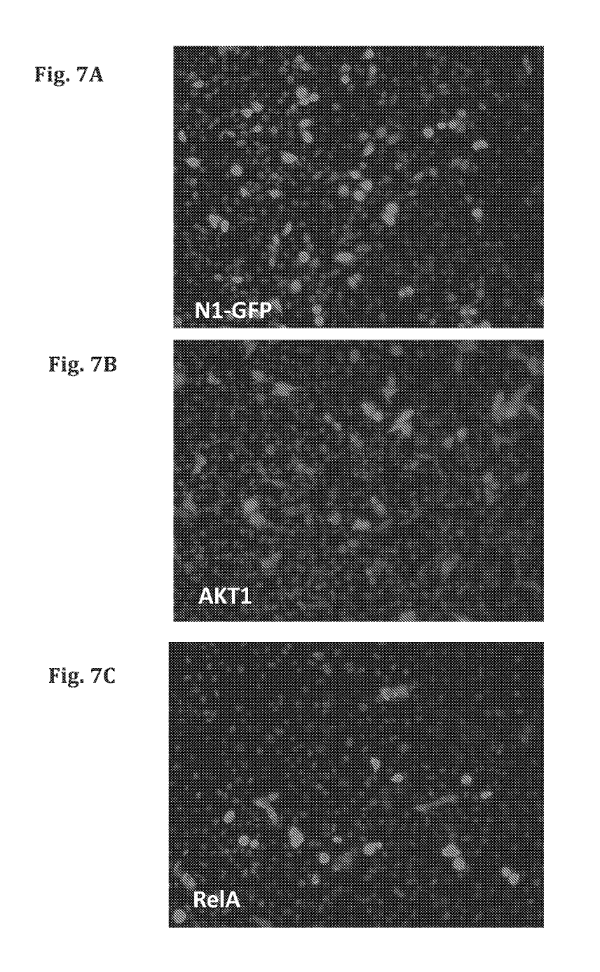

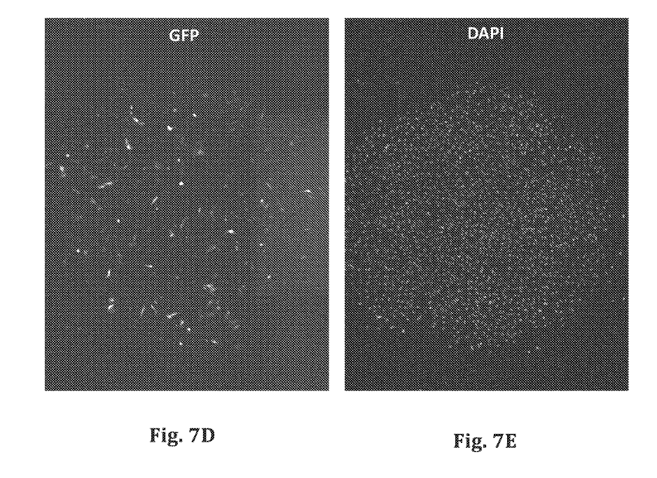

The results presented in FIGS. 7A-C, show pictogram of Hela cells manually seeded on top a poly-l-lysine coated slide and manually transfected via the indirect transfection process with expression vectors encoding for the following proteins: GFP (FIG. 7A), AKT1 conjugated to GFP (FIG. 7B), RelA conjugated to GFP (FIG. 7C). All cells in the image are labeled with DAPI dye staining the nuclei. Fluorescent cells are cells those which underwent transfection.

The results presented in FIGS. 7D-E show images of cells in a single spot (chamber) on the array generated using automatic tooling. FIG. 7D shows fluorescent cells, which are cells that underwent transfection. FIG. 7E shows DAPI staining of the nucleic of the cells in the same spot. The spot/chamber was transfected simultaneously with KRAS and GFP-conjugated ERK2 expression vectors, GFP is used as a fluorescent marker. The images are of 10× magnification; 4×4 image stitching.

### Example 3: Antibody Printing and Cell-Surface Marker Screen

Exemplified herein is a printing method for a mesh carrying separable sets of different antibodies directed against cell-surface markers in an array pattern and its use for parallel screening of surface markers expression in live cells.

According to the example, a mesh is stretched over a dedicated frame. The mesh in this example is a 60 μm nylon mesh (for example Merck Millipore, cat no. NY6000010). The mesh is patterned by vertical and horizontal 1 mm thick lines of liquid, thermoplastic, polymeric, non-toxic hydrophobic material with 1 mm² chambers (printing spaces/spots) confined by the vertical and horizontal hydrophobic lines resulting in a 2 mm pitch. The mesh is then baked for a duration of 20 min in an oven pre-heated to 100° C. to solidify the polymer pattern.

A panel of antibodies against different cell surface markers, all fluorescently labeled, in a suitable 0.5 mg/ml solution, are dispensed/dripped onto different chambers of the gridded mesh, in an addressable manner, after verifying alignment of the dispenser and the array on the mesh. Dispensing may be performed manually or using an automated or semi-automated dispenser.

The printing may be performed in a humid environment (in a climate controlled chamber) in order to prevent the antibody-containing solutions from drying. After printing, the mesh-holding frame is transferred to humidity chambers until used.

Prior to approximating the antibody mesh and the cells-carrying slide, the cell slide is prepared for transfection by eliminating most fluids from the carrying case except for fluids retained by the cells themselves, for example, by using vacuum-based aspiration. Then, the mesh-holding frame is aligned and place over the cells on the substrate, by aligning the frame and the substrate casing, such that each printed chamber is located above a seeded spot; printed side of the mesh faced down. Then, 0.3 μl of blocking solution is added above each chamber, followed by 45 minutes of incubation at 37° C.

To separate the mesh from the slide, the slide-mesh interface is flooded with 2-3 ml PBS solution to cancel out capillary forces fastening the mesh to the slide. Then the mesh frame (with the mesh) is gently lifted from the substrate.

PBS solution is added to immerse the top surface of the slide.

The labeled cells may then be optionally fixed to the surface of the slide for preservation and for further processing and analysis in downstream assays.

The resulting array can be used to identify the list of surface markers expressed by the specific cell population deposited on the slide: different and specific cell spots on the array will be fluorescently labeled according to the specific antibody they have been exposed to and according to whether or not the respective surface marker is expressed by the deposited cells.

1. A system for transfection of nucleic acid molecules, the system comprising:
a transparent substrate having a surface suitable for attachment of viable cells;
a plurality of viable cells deposited on the surface in an array of predetermined locations that are not contiguous to one another;
a mesh suitable for retention of nucleic-acid molecules; and
nucleic-acid molecules deposited in a designated array on said mesh,
wherein said mesh is configured to be placed in alignment above the cells on said surface such that the interface between the mesh and the cells on said surface enables release of at least some of said nucleic-acid molecules, thereby transfecting at least some of said viable cells.

2. The system of claim 1, wherein said nucleic-acid molecules comprise multiple nucleic-acid molecule sets arranged in the designated array on said mesh, such that said nucleic-acid molecule sets are separated from each other, wherein said mesh is configured to be placed above the cells on said surface and release at least some of said nucleic-acid molecule sets, thereby transfecting at least some of said viable cells, while maintaining spatial separation of the sets within the designated array.

3. (canceled)

4. The system of claim 1, wherein said mesh is comprised of a polymeric material.

5. The system of claim 1, wherein said viable cells are homogeneously deposited within perimeters of the predetermined locations on said surface.

6. (canceled)

7. The system of claim 2, wherein said substrate surface is suitable for attachment of a plurality of viable cell groups, wherein said cell groups are separated from each other, wherein locations of said viable cell groups correspond to locations of said nucleic-acid molecule sets such that upon approximating said mesh and said cell groups on the surface, at least some nucleic-acid molecule sets are introduced to at least some corresponding viable cell groups, such that a specific nucleic-acid molecule set is introduced to a single viable cell group.

8. The system of claim 2, wherein said mesh comprises a grid configured to define the separation between said nucleic-acid molecule sets within the designated array.

9. The system of claim 8, wherein said substrate comprises a grid configured to define the separation between said viable cell groups.

10. The system of claim 8, wherein said grid comprises a non-toxic hydrophobic material.

11. (canceled)

12. The system of claim 1, further comprising a nucleic-acid molecule dispenser configured to dispense said nucleic-acid molecules sets onto said mesh.

13. The system of claim 1, further comprising a frame, configured to facilitate alignment of said substrate and said mesh, wherein said frame is configured to enable separation between said mesh and said substrate, essentially without affecting the cells.

14. (canceled)

15. A kit for transfection of nucleic acid molecules, the kit comprising:
a mesh deposited with nucleic-acid molecules sets arranged in a designated array defined by a grid on said mesh, wherein said nucleic-acid molecules sets are separated from each other by said grid, wherein the grid maintains spatial separation of the sets within the designated array;
wherein said mesh is configured to be placed above a substrate surface bearing viable cells arranged in a predetermined pattern and to release at least some of the nucleic-acid molecules to be transfected to at least some of the viable cells, while maintaining spatial separation of the sets within the designated array, wherein the designated array aligns with at least part of the pattern of the target cells.

16. (canceled)

17. The kit of claim 15, wherein said mesh is a comprised of a polymeric material.

18. The kit of claim 15, wherein said grid comprises a non-toxic hydrophobic material.

19. (canceled)

20. The kit of claim 15, further comprising a transparent substrate having a surface configured for attachment of viable cells and/or a frame for maintaining the orientation of the mesh in alignment with the substrate surface bearing the viable cells.

21. The kit of claim 20, wherein the frame is configured to enable detachment of said mesh from the substrate surface.

22. The kit according to claim 15, further comprising a transfection agent deposited with the nucleic-acid molecules.

23. A method for transfecting nucleic-acid molecules into cells, the method comprising:
providing a mesh comprising nucleic-acid molecules deposited on said mesh in a designated array;
providing a substrate comprising a surface deposited with viable cells in an array of predetermined locations; and
approximating a surface of the mesh to the viable cells on the substrate surface such that at least some nucleic-acid molecules are introduced to at least some of the viable cells, thereby transfecting at least some of the viable cells.

24. The method of claim 23, wherein the nucleic-acid molecules comprise a plurality of nucleic-acid molecule sets deposited on said mesh in a designated array, such that said nucleic-acid molecule sets are separated from each other, wherein upon approximating the surface of the mesh to the cells on the substrate surface at least some nucleic-acid molecule sets are transfected into at least some of the viable cells, while maintaining spatial separation of the sets within the designated array.

25. The method according to claim 23 wherein the mesh comprises a grid that maintains spatial separation between the nucleic acid molecule sets within the designated array.

26. The method of claim 23, wherein the nucleic-acid molecules are deposited on the mesh in the presence of a transfection reagent.

27. The method of claim 23, wherein approximating the surface of the mesh to the viable cells on the substrate surface is performed in the presence of an aqueous solution.

28. (canceled)

29. The method of claim 23, wherein the viable cells are divided to a plurality of groups, wherein said groups are separated from each other.

30. The method of claim 24, wherein the viable cells are divided to a plurality of groups, wherein said groups are separated from each other, wherein locations of said viable cell groups correspond to locations of said nucleic-acid molecule sets, such that upon approximating said mesh to said cells on the surface, at least some nucleic-acid molecule sets are transfected to at least some corresponding viable cell groups, such that a specific nucleic-acid molecule set is introduced to a specific viable cell group.

31. The method of claim 24, further comprising dispensing the plurality of nucleic-acid molecule sets on the mesh according to the designated array.

32. (canceled)

33. The method of claim 23, further comprising:
incubating the viable cells on the substrate with the mesh; and
separating the mesh from the cells on the substrate, thereby obtaining a substrate with multiple sets of transfected cells.

34.-41. (canceled)
